(19) United States
(12) Patent Application Publication
(10) Pub. No.: US 2007/0148712 A1
(43) Pub. Date: Jun. 28, 2007

(54) Breast cancer and prostate cancer assessment

(75) Inventors: Iouri Y. Markouchine, Manhattan, KS (US); Ryszard Jankowiak, Manhattan, KS (US); Ercole Cavalieri, Waterloo, NE (US); Muhammad Saeed, Omaha, NE (US); Eleanor Rogan, Omaha, NE (US)

Correspondence Address: BARNES & THORNBURG LLP, P.O. BOX 2786, CHICAGO, IL, 60690-2786, US

(73) Assignees: Iowa State University Research Foundation, Inc., Ames, IA (US); Board of Regents of the University of Nebraska, Lincoln, NE (US)

(21) Appl. No.: 11/450,808

(22) Filed: Jun. 8, 2006

Related U.S. Application Data
(60) Provisional application No. 60/688,535, filed on Jun. 8, 2005.

(51) Int. Cl. G01N 33/574; C12M 1/34

(52) U.S. Cl. 435/7.23; 435/287.2; 530/391.1; 977/902

(57) ABSTRACT

A method for detecting a biological marker in a sample, which comprises a complex mixture of molecules, from a patient comprising exposing a detection site having bound monoclonal antibodies specific for the biological marker to the sample, exposing the detection site to a detectably labeled reporter molecule, which is substantially identical to the biological marker, and assessing the degree of binding at the detection site by the reporter molecule; reporter molecules; haptens; and monoclonal antibodies.

### CROSS-REFERENCE TO RELATED PATENT APPLICATIONS

This application claims priority to U.S. Provisional Pat. App. No. 60/688,535, which was filed on Jun. 8, 2005, and is hereby incorporated by reference.

### STATEMENT REGARDING FEDERALLY SPONSORED RESEARCH AND DEVELOPMENT

The present invention was developed, at least in part, with funding from the National Institutes of Health through Iowa State University Program Project Grant P01 CA49210. Therefore, the government of the United States of America may have certain rights in the invention.

### FIELD OF THE INVENTION

The present invention relates to a method for detecting in a sample a molecule, in particular an estrogen-derived conjugate, which serves as a biological marker in risk assessment of breast and prostate cancers. The present invention also relates to materials for use in such a method.

### BACKGROUND OF THE INVENTION

Estrogens are associated with several cancers in humans and are known to induce tumors in rodents (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol. 382, Elsevier, Düsseldorf, Germany, 293-319 (2004)). Estrone (E₁) and estradiol (E₂) are obtained by aromatization of androstenedione and testosterone, respectively, catalyzed by cytochrome P450 (CYP)19, aromatase (Jefcoate, C. R. et al., Tissue-specific synthesis and oxidative metabolism of estrogens. In: JNCI Monograph: Estroens as Endogenous Carcinogens in the Breast and Prostate, No. 27, (E. Cavalieri and E. Rogan, Eds.), pp. 95-111. Oxford University Press, Maryland (2000)). E₁ and E₂ are biochemically interconvertible by 17β-estradiol dehyrogenase; their metabolism leads to catechol estrogens and, to a lesser extent, 16□-hydroxylation (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol.382, Elsevier, Düsseldorf, Germany, 293-319 (2004)). The catechol estrogens formed are 2-hydroxyE₁(E₂) [2-OHE₁(E₂)] as the major one and 4-OHE₁(E₂) as the minor one (Guengerich, Annu. Rev. Pharmacol. Toxicol. 29, 241-264 (1989); Martucci et al., Pharmacol. Ther. 57, 237-257 (1993); Zhu et al., Carcinogenesis 19, 1-27 (1998)). In general, these two catechol estrogens are inactivated in the liver by conjugative reactions, such as glucuronidation, sulfation, and O-methylation. In extrahepatic tissues, the major pathway of conjugation occurs by O-methylation catalyzed by the ubiquitous catechol-O-methyltransferase (COMT) (Mannisto et al., Pharmacol. Rev. 51, 593-628 (1999)). The level and/or induction of CYP1B1 (Savas et al., J Biol. Chem. 269, 14905-14911 (1994); Hayes et al., Proc. Natl. Acad. Sci. USA 93, 9776-9781 (1996); Spink et al., Carcinogenesis 19, 291-298 (1998)) and other 4-hydroxylases could render 4-OHE₁(E₂) as the major metabolite, rather than the usual 2-OHE₁(E₂). In this case, conjugation of 4-OHE₁(E₂) by methylation in extrahepatic tissues might become insufficient, and competitive catalytic oxidation of catechol estrogens to catechol estrogen quinones (CEQ) could occur (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol. 382, Elsevier, Düsseldorf, Germany, 293-319 (2004)).

Catechol estrogen quinones (CEQ) can be neutralized by reaction with glutathione (GSH). A second inactivating pathway for CEQ is their reduction to catechol estrogens by quinone reductase and/or cytochrome P450 reductase (Emester et al., Chemica Scripta 27A (1987); Roy et al., J Biol. Chem. 263, 3646-3651 (1988)). If these two inactivating processes are insufficient, CEQ may react with DNA to form stable and depurinating adducts (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol. 382, Elsevier, Düsseldorf, Germany, 293-319 (2004); Li et al., Carcinogenesis 25, 289-297 (2004)). The carcinogenic 4-OHE₁(E₂) (Liehr et al., J Steroid Biochem. 24, 353-356 (1986); Li et al., Fed. Proc. 46, 1858-1863 (1987); Newbold et al., Cancer Res. 60, 235-237 (2000)) are oxidized to form predominantly the depurinating adducts 4-OHE₁(E₂)-1-N3Ade and 4-OHE₁(E₂)-1-N7Gua (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol. 382, Elsevier, Düsseldorf, Germany, 293-319 (2004); Li et al., Carcinogenesis 25, 289-297 (2004); Cavalieri et al., Proc. Amer. Assoc. Cancer Res. 44, 180 (2003)), whereas the borderline carcinogenic 2-OHE₁(E₂) (Liehr et al., J Steroid Biochem. 24, 353-356 (1986); Li et al., Fed. Proc. 46, 1858-1863 (1987); Newbold et al., Cancer Res. 60, 235-237 (2000)) are oxidized to form much lower levels of the depurinating adducts 2-OHE₁(E₂)-6-N3Ade and higher levels of stable adducts than 4-OHE₁(E₂) (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol.382, Elsevier, Düsseldorf, Germany, 293-319 (2004); Li et al., Carcinogenesis 25, 289-297 (2004); Cavalieri et al., Proc. Amer. Assoc. Cancer Res. 44, 180 (2003)). It is the imbalance between activating pathways and protective pathways that can trigger a substantial reaction of EI(E₂)-3,4-Q with DNA (Cavalieri et al., Chem. Res. Toxicol. 14, 1041-1050 (2001); Rogan et al., Carcinogenesis 24, 697-702 (2003)), thereby initiating mutations that can lead to cancer (Chakravarti et al., Oncogene 20, 7945-7953 (2001)).

Formation of CEQ-derived GSH (γ-glutamyl-L-cysteinylglycine) conjugates has already been demonstrated in in vivo experiments (Cavalieri et al., Chem. Res. Toxicol. 14, 1041-1050 (2001); Rogan et al., Carcinogenesis 24, 697-702 (2003); Devanesan et al., Carcinogenesis 22, 489-497 (2001); Todorovic et al., Carcinogenesis 22, 905-911 (2001); Devanesan et al., Carcinogenesis 22, 1573-1576 (2001)). These conjugates are considered to be potentially useful biomarkers for catechol estrogen-induced DNA damage and risk of breast and other cancers. Conjugation with GSH prevents damage to DNA (Cavalieri et al., Chem. Res. Toxicol. 14, 1041-1050 (2001)), which is one effect of this important detoxification pathway in biological systems. A large number of electrophilic compounds conjugate with GSH nonenzymatically or, more effectively, via S-transferase-catalyzed reactions (Cavalieri et al., The role of endogenous catechol quinones in the initiation of cancer and neurodegenerative diseases. In: Methods in Enzymology, Quinones and Quinone Enzymes, Part B (H. Sies & L. Packer, Eds.), Vol.382, Elsevier, Düsseldorf, Germany, 293-319 (2004); Cao et al., Chem. Res. Toxicol. 11, 917-924 (1998)). Therefore, the reaction of CEQ with various sulfur nucleophiles, RSH, in which R is the cysteine (Cys), N-acetylcysteine (NAcCys), or GSH moiety, is of great interest in carcinogenesis. Once CEQ conjugates are formed, catabolism occurs via mercapturic acid biosynthesis. First, the glutamyl moiety of the GSH conjugate is removed by transpeptidation, catalyzed by γ-glutamyl transpeptidase. Then the cysteinylglycine derivative is hydrolyzed to yield the Cys conjugate. The final step consists of acetylation to the NAcCys conjugate and excretion in urine (Cavalieri et al., The role of endogenous catechol quinones in the initiation of cancer and neurodegenerative diseases. In: Methods in Enzvmology, Quinones and Quinone Enzymes, Part B (H. Sies & L. Packer, Eds.), Vol. 382, Elsevier, Düsseldorf, Germany, 293-319 (2004); Todorovic et al., Carcinogenesis 22, 905-911 (2001); Nakagomi et al., Chem. Res. Toxicol. 13, 1208-1213 (2000)). Therefore, identification and quantitation of CEQ conjugates in urine has potential for assessing the level of CEQ formed. Schematic structures of 4-OHE₁, 4-OHE₂, and 4-OHE₁(E₂)-NAcCys conjugates are shown in FIG. 1.

Recent analysis of potential biomarkers of estrogen-initiated cancer in urine and the kidney of Syrian golden hamsters treated with 4-OHE₂ revealed that HPLC with electrochemical detection (with picomole detection limit) provides high specificity (Cavalieri et al., Chem. Res. Toxicol. 14, 1041-1050 (2001); Devanesan et al., Carcinogenesis 22, 489-497 (2001), Todorovic et al., Carcinogenesis 22, 905-911 (2001)). Nagakomi and Suzuki developed a protocol for the quantitation of NAcCys conjugates in the urine of rats and hamsters using an immunoaffinity column (Nakagomi et al., Chem. Res. Toxicol. 13, 1208-1213 (2000)). Recently, to improve the detection limit of CEQ-derived conjugates, spectrophotometric monitoring was investigated (Jankowiak et al., Chem. Res. Toxicol. 16, 304-311 (2003)). It was shown that: i) 4-OHE₁ and 4-OHE₂-derived NAcCys conjugates are weakly fluorescent at 300 K (with emission maximum at 332 nm), but strongly phosphorescent at 77 K; ii) Cys and NAcCys exhibit fluorescence and phosphorescence only at 77 K; and iii) 4-OHE₁, and 4-OHE₂ are weakly fluorescent at 300 and 77 K and not phosphorescent. The phosphorescence spectra of NAcCys conjugates are characterized by a weak origin band at ˜383 nm and two intense vibronic bands at 407 and 425 nm. Upon cooling from 300 to 77 K, the total luminescence intensity of SG and NAcCys conjugates increases by a factor of ˜150, predominantly due to phosphorescence enhancement. Theoretical calculations revealed, in agreement with the expenmental data, that the lowest singlet (S₁) and triplet (T₁) states of 4-OHE₂-2-NAcCys are of n,π^{*} and π,π^{*} character, respectively, leading to a large intersystem crossing yield and strong phosphorescence. The limit of detection (LOD) for CEQ-derived conjugates, based on phosphorescence measurements, is in the low femtomole range. The concentration LOD is approximately 10⁻⁹ M (Jankowiak et al. Chem. Res. Toxicol. 16, 304-311 (2003)). Therefore, it has been proposed that capillary electrophoresis (CE) interfaced with low temperature phosphorescence detection can be used to test human exposure to CEQ by analyzing urine.

In view of the above, the present invention seeks to provide materials and methods with improved sensitivity and case of use for the detection of CEQ-derived conjugates, such as in the assessment of breast and prostate cancers. The methodology has wider application, particularly for molecules and haptens that are too small to allow the use of conventional methods of screening, e.g., detectably labeled secondary antibodies. This and other objects and advantages of the present invention, as well as additional inventive features, will become apparent to those of ordinary skill in the art upon reading the detailed description set forth herein.

### SUMMARY OF THE INVENTION

The present invention provides a method for detecting a biological marker in a sample, which comprises a complex mixture of molecules, from a patient. The method comprises exposing a detection site having bound monoclonal antibodies (MAb) specific for the biological marker to the sample; washing the detection site with a solution that removes substantially unbound molecules from the detection site; exposing the detection site to a detectably labeled reporter molecule, which is substantially identical to the biological marker; washing the detection site with a solution that removes substantially unbound molecules from the detection site; and assessing the degree of binding at the detection site by the reporter molecule, wherein a high degree of binding by the reporter molecule is indicative of an absence or a low concentration of the biological marker in the sample, and wherein an absence or a low degree of binding by the reporter molecules is indicative of a high concentration or moderate concentration of the biological marker in the sample.

The present invention also provides a reporter molecule selected from the group consisting of a 4-OHE₁-2-N-acetylcysteine(NAcCys) conjugate, a 4-OHE₂-2-NAcCys conjugate, a 4-OHE₁-1-N3 adenine (Ade) adduct, and a 4-OHE₂-1-N3 Ade adduct, and wherein the reporter molecule is detectably-labeled. The present invention also provides a monoclonal antibody having specificity for 4-OHE₁-2-N-AcCys and 4-OHE₂-2-NAcCys conjugate molecules; and a monoclonal antibody having specificity for 4-OHE1-1-N3Ade and 4-OHE2-1-N3Ade adducts.

In view of the above, the present invention also provides a biochip comprising a monoclonal antibody having specificity for a conjugate and/or a DNA adduct derived from CEQ. Additionally, a kit comprising the biochip, a hapten, and a reporter molecule is provided.

### BRIEF DESCRIPTION OF THE DRAWINGS

FIG. 1 is a diagram depicting the chemical structures of 2-OHE₁(E₂), 4-OHE₁(E₂), and 4-OHE₁(E₂)-2-NAcCys.

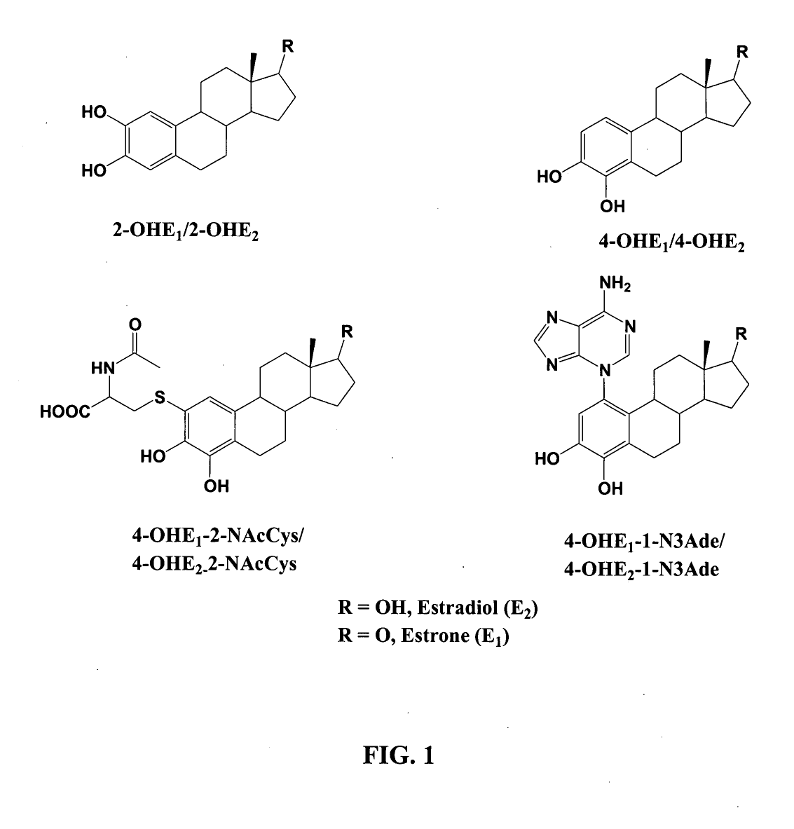

FIG. 2 is a diagram depicting synthesis of the 4-OHE₁(E₂)-2-NAcCys-16-MCC hapten (6): (1) 4-OHE₁; (2) 4-OHE₁-2-NAcCys; (3) 4-O-TBDMS-E₁-2-NAcCys; (4) 4-O-TBDMS-E₁-2-NAcCys enolate; (5) 4-O-TBDMS-E₁-2-NAcCys-16-MCC; (6) 4-OHE₁-2- NAcCys-16-MCC.

FIG. 3 is a diagram depicting synthesis of the 4-OH-17aminomethyl(AM)E₂-2-NAcCys-MCC hapten (12): (1) 4-OHE₁; (7) 3,4-isopropylidene-E₁; (8) 3,4-isopropylidene-17-nitrile-17-O-trimethylsilyl-E₂; (9) 3,4-isopropylidene-17-AM-E₂; (10) 4-OH-17-AM-E₂; (11) 4-OH-17-AM-E₂-2-MAcCys; (12) 4-OH-17-AM-E₂-2-NAcCys-MCC.

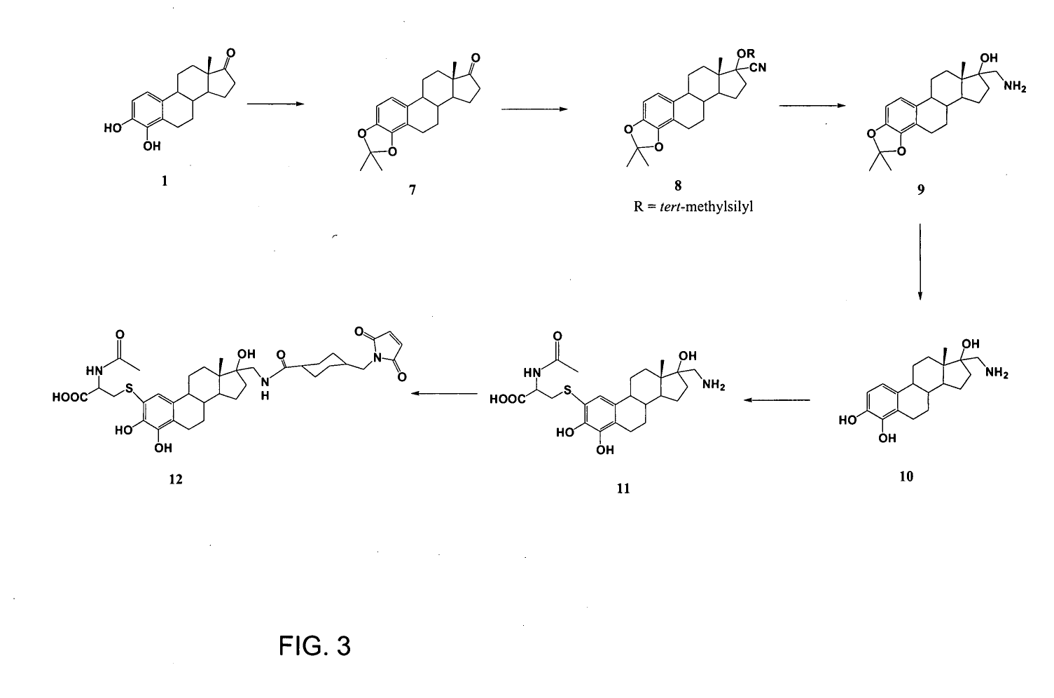

FIG. 4 is a diagram depicting synthesis of the 4-OH-17-AME₂-1- N3Ade-MCC hapten (14): (10) 4-OH-17-AM-E₂; (13) 4-OH-17-AM-E₂-1-N3Ade; (14) 4-OH-17-AM-E₂.

FIG. 5 is a diagram depicting inhibition profiles obtained for 4-OHE₁-2-NAcCys (curve 1); NAcCys (curve 2), 4-OHE₁(E₂) (curve 3), and 4-OHE₁-1-N3Ade (curve 4) using the 2E9 MAb in the competitive ELISA assay. Competitor-mediated reduction of MAb binding was expressed as % inhibition vs. untreated MAb and then plotted as a function of log quantity of competitor per well of the ELISA plate.

FIG. 6 is a diagram depicting the results of two CE electropherograms: Curve A is the CE electropherogram (observation wavelength at 214 nm); peaks 1, 2, 3, and 5 correspond to 4-OHE₁-1-N3Ade, 4-OHE₁, and 4-OHE₂, and NAcCys, respectively (concentration, c=10⁻⁶ M). Peak 4 (near 5 min migration time labeled by a solid arrow) corresponds to the 4-OHE₁-2-NAcCys at a significantly lower concentration (i.e., 10⁻⁸ M). Curve B is the CE electropherogram obtained for the same mixture passed through the 2E9 MAb-based affinity column and pre-concentrated by a factor of 100. The major peak 4 corresponds to the captured and highly concentrated 4-OHE₁-2-NAcCys conjugate.

FIG. 7 is a diagram depicting curves A and B, i.e., the room temperature (300 K) (multiplied by a factor of 5) and 77 K luminescence spectra of the 4-OHE₁-2-NAcCys, respectively. Both spectra were obtained in glycerol/H₂O glass (10 mM phosphate buffer) at pH=3 with excitation wavelength (λₑₓ) of 257.0 nm.

FIG. 8 is a diagram depicting the specificity of the 2E9 MAb raised against 4-OHE₁-2-NAcCys. The bars show relative phosphorescence intensity obtained for the first three fractions of 4-OHE₁-2-NAcCys eluted from the immunoaffinity column. The amount of spiked, buffered urine sample run through the column was 1 mL (c=4×10⁻⁷ M) and 100 mL (c=4×10⁻⁹ M) for frames A and B, respectively.

FIG. 9 is a diagram depicting the results of electropherograms: Curve a: CE electropherogram of a mixture of four analytes in a buffer solution; peaks 1, 2, 3, and 4 correspond to 4-OHE₁-1-N3Ade, 4-OHE₁-2-NAcCys, 4-OHE, and 4-OHE₁-1-N7Gua, respectively. Curve b: electropherogram of a phosphate-buffered saline (PBS) sample spiked with analytes 1-4 listed above and run through the affinity column [only 4-OHE₁-2-NAcCys (peak 2) was recovered]. Curve c: CE electropherogram obtained after a diluted human urine sample was spiked with 4-OHE₁-2-NAcCys and run through the affinity column. Peak 2 reveals an excellent recovery of 4-OHE₁-2-NAcCys. Curve d: a electropherogram of 4-OHE₁-2-NAcCys standard.

FIG. 10 is a diagram depicting a method in accordance with an embodiment of the present invention, including an MAb-based biosensor with multiple active spots on a chip surface.

FIG. 11 is a diagram depicting instrumentation for on-chip analysis. S1 and S2 label the active area on the chip surface. Thus, the system will provide video image capture, processing, analysis, quantitation, spectroscopic characterization and calibration curves for adducts of interest.

FIG. 12 is a diagram depicting a MAb-based biosensor using the method of FIG. 11.

FIG. 13 is a diagram depicting a calibration curve for 4-OHE₂-2-NAcCys conjugate using a chip with multiple active spots. Detection is based on emission of 4-OHE₂-2-NAcCys conjugate derivatized with SAMSA via a succinimidyl 4-(N-maleimido-methyl)cyclohexane-1-carboxylate (SMCC).

FIG. 14 is a diagram depicting the derivatization of haptens (6), (12) and (14) with flurophore SAMSA via SMCC.

FIG. 15 is a diagram depicting the labeling of 4-OHE₂-2-NAcCys conjugate with quantum dots.

FIG. 16 is a diagram depicting identification of 4-OHE₁-2-NAcCys (peak 1) and 4-OHE₁-1-N3Ade (peak 2) in human urine from a woman with breast carcinoma (see text).

FIG. 17 is a diagram depicting detection of 4-OHE₁(E₂)-1-N3Ade in three human urine samples labeled as B-1, E-1, and M-1. The bars correspond to the integrated (normalized) area of the electropherogram peak assigned to the 4-OHE₁(E₂)-1-N3Ade. B. 77K luminescence spectra (F=fluorescence; P=phosphorescence) of the 4-OHE₁(E₂)-1-N3Ade; both spectra were obtained in glycerol/buffer glass (10 mM phosphate buffer) at pH=2 with excitation wavelength of 257.0 nm.

FIG. 18 is a diagram depicting the detection and identification of 4-OHE₁(E₂)-1-N3Ade in human urine samples.

### DETAILED DESCRIPTION OF THE INVENTION

The present invention provides a method for detecting a biological marker in a sample, which comprises a complex mixture of molecules, from a patient. The method comprises exposing a detection site, which has bound MAbs that are specific for the biological marker, to the sample; washing the detection site with a solution that removes substantially unbound molecules, which were present in the complex mixture of molecules, from the detection site; exposing the detection site to a detectably labeled reporter molecule, which is substantially identical to the biological marker; washing the detection site with a solution that removes substantially unbound molecules (i.e., unbound detectably labeled reporter molecules) from the detection site; and assessing the degree of binding at the detection site by the reporter molecules. A high degree of binding by the reporter molecule is indicative of an absence or a low concentration of the biological marker in the sample, whereas an absence or a low degree of binding by the reporter molecule is indicative of a high concentration or moderate concentration of the biological marker in the sample.

By “sample” is meant any biological sample that can be subjected to the method. Examples of samples include, but are not limited to, urine, serum, and nipple aspirate fluid.

By “biological marker” is meant a molecule that is correlated with the presence of or the risk for disease. An example is a conjugate derived from CEQ, such as 4-OHE₁ and 4-OHE₂ conjugates.

By “detection site” is meant any suitable substrate as is known in the art to which MAbs can be bound. The binding of MAbs to such substrates is within the ordinary skill in the art, as are the exposing of such substrates to molecules and the washing of such substrates to remove unbound and substantially unbound molecules. Solutions and conditions of the exposing and washing steps are exemplified herein.

The present invention also provides a reporter molecule selected from the group consisting of a 4-OHE₁-2-NAcCys conjugate, a 4-OHE₂-2-NAcCys conjugate, a 4-OHE₁-1-N3 Ade adduct, and a 4-OHE₂-1-N3 Ade adduct. The reporter molecule is detectably labeled. Any suitable label can be used, such as, for example, a fluorophore, a chromophore, a radionuclide, or other fluorescent marker (e.g., quantum dot). Such reporter molecules can be synthesized and labeled in accordance with any suitable method known in the art and as exemplified herein.

The present invention also provides an MAb having specificity for 4-OHE₁-2-NAcCys conjugate and 4-OHE₂-2-NAcCys conjugate conjugates; and an MAb having specificity for 4-OHE₁-1-N3 Ade adduct and 4-OHE₂-1-N3 Ade adducts. The production (raising) of MAbs is known in the art (see, e.g., Lo (2004) and Harlow (2001) under “EXAMPLES”) and is exemplified herein. In this regard, the present invention also provides haptens for the generation of MAbs against 4-OHE₁-2-NAcCys and 4-OHE₂-2-NAcCys. In particular, the present invention provides 4-OHE₁-2-NAcCys-16-MCC, 4-OH-17AM-E₂-2-NAcCys, and 4-OH-17-AME₂-1-N3Ade, which can be optionally labeled with a detectable label, such as a fluorescent label, e.g., SAMSA or quantum dots.

The present invention also provides a novel and efficient room temperature method and device for screening estrogen-derived conjugates that serve as biomarkers in the risk assessment of breast and prostate cancers. The device utilized in one embodiment of the present invention is an MAb-based biosensor (biochip) on any suitable substrate (e.g., glass, polymer, and/or silicon wafer substrates) having multiple addressable patches on the surface, designed and built for sensitive and selective detection of CEQ-derived biomarkers. Detection in the biochips can be based on a “first-come-first-served” approach and can employ fluorescence-based imaging. The methodologies discussed below can be used, for example, in the context of cost-effective, high-throughput screening and future cancer risk assessment. A calibration curve for the detection of CEQ-derived conjugates has been established. The device and method of the present invention can be used to study any patient sample, such as, for example, urine, serum, nipple aspirate fluid and/or tissue extracts obtained from human breast cancer patients and prostate cancer patients.

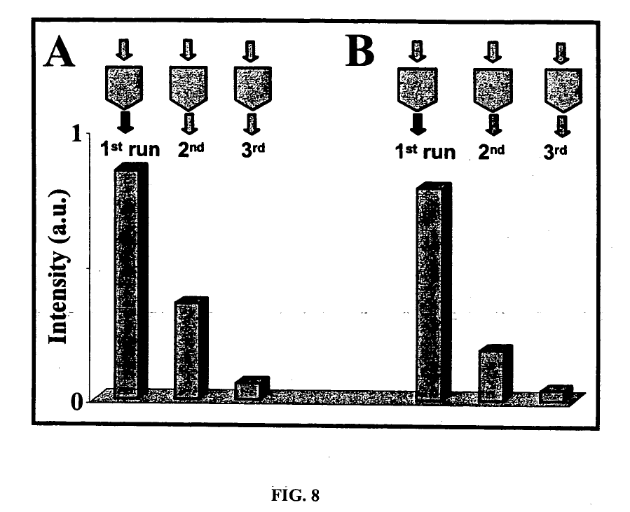

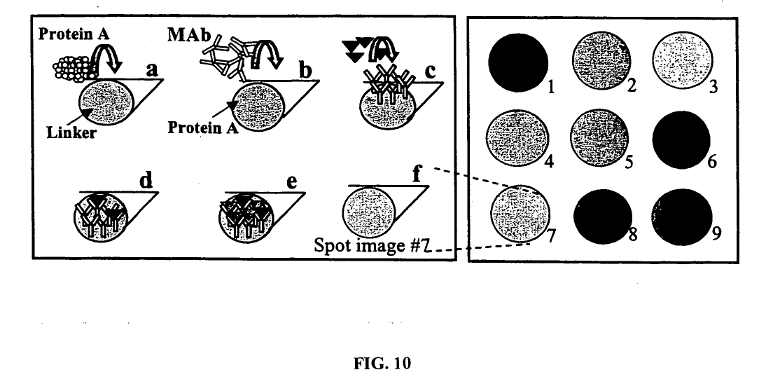

The concept of surface-based biochips using the “first-come-first-served” approach is graphically illustrated in FIG. 10. The principles are illustrated for a single sensing area derivatized with a specific MAb, but biochips with multiple addresses for simultaneous detection of several analytes of interest, as well as those with a single sensing area and multiple MAbs can be developed. The biochips can be built in the form of microarrays with an nxm architecture, where n and m correspond to the number of rows and columns, respectively. The left frame of FIG. 8 illustrates preparation of the MAb-based sensing area of a biochip in accordance with an embodiment of the present invention. For simplicity, a single spot is considered. Dithiobis(succinimidyl propionate) (DSP) can be used as a linker, since it binds more protein than dithiobis(succinimidyl undecanoate) (DSU). The following steps include: (a) immobilization of protein A; (b) binding of MAb that is specific for the analyte of interest, say A₁; (c) exposure to a mixture of analytes that might include A₁; (d) washing to remove unbound analytes and/or analytes with short dissociation times; (e) read-out procedure, wherein the spot is exposed to an excess A₁ labeled with 5-((2-(and-3)-S-(acetylmercapto)succinoyl)amino)fluorescein (SAMSA) (i.e., A₁*) (or any other suitable detectable label, such as, for example, a fluorophore, a chromophore, a radionuclide, or other fluorescent marker, e.g., quantum dot), and then washed again; and finally (f) a fluorescence-based image of the spot is acquired. Of course, as discussed above, the more A₁ on the biosensor surface, the darker the resulting image, as A₁ does not fluoresce. For example, on the 3 by 3 array with 9 active spot areas shown on the right side of FIG. 10, spots 1 and 6 correspond to a high concentration of A₁, whereas spots 2-5 and 7-9 contain smaller and different amounts of A₁. Thus, the above-described method can provide means for identifying and quantitating biomarkers for which specific MAbs are available. In this case, a sub-femtomole LOD with an excellent dynamic range can be achieved. These Au/DSP/protein A/MAb nanoassemblies, with single and/or multiple sensing patches, will be suitable for detection and quantitation of various CEQ-derived conjugates.

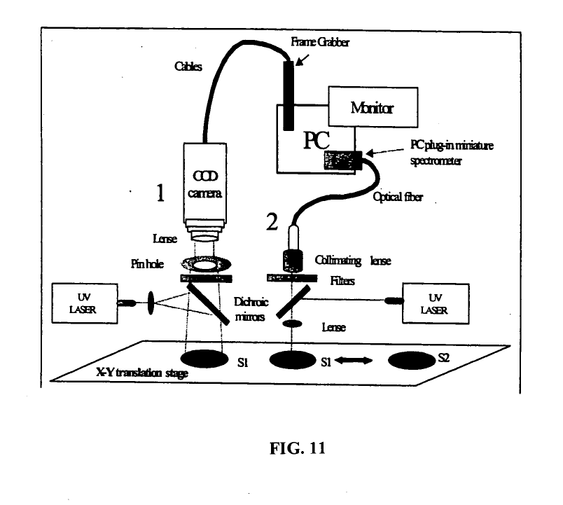

Sensitive fluorescence-based imaging enables cost-effective identification of biomarkers, such as estrogen-derived biomarkers, in clinical applications. Proposed instrumentation is shown in FIG. 11. The apparatus can consist of a laser (or ultraviolet lamp), a CCD camera to provide 3-D plots of integrated luminescence intensity and images, a miniature PC plug-in spectrometer to acquire spectra; specially designed optics; a sample translation module; and software for spectral integration and imaging.

The utility of MAbs for detecting and isolating antigens (or haptens) can hardly be overstated, given the wide applications developed over the years. Typically, antibodies are chemically tagged with fluorescent, magnetic, radioactive, and assorted other compounds as a way of facilitating antigen detection or isolation under a variety of expenmental conditions. The MAb or antigen/hapten to be detected does not have to be tagged. The detection of biomarkers can be performed in a “label-free-fashion.” The read-out procedure still employs derivatized standards. Examples of such an approach include surface-based biosensors with several active sensing areas and transparent affinity columns. In both cases the detection is based on fluorescence imaging.

The equilibrium constant and the kinetic off- and on-rates for the analyte and the reporter molecule of interest must be known. The equilibrium association constants (K_{A}) and association (kₒₙ)/dissociation (k_{off}) rates for 4-OHE₂-2-NAcCys and 4-OHE₂-2-NAcCys-SAMSA conjugates are given in Table I, which also provides information on other closely related analytes, such as 4-OHE₂, NAcCys, and 4-OHE₂-1-N3Ade adduct. All values listed in Table I were determined using an indirect competitive ELISA. The value of K_{A} for 4-OHE₂-2-NAcCys-SAMSA was confirmed by an equilibrium dialysis method.

TABLE I
The equilibrium association constants (K_{A}) and association
(kₒₙ)/dissociation (k_{off}) rates for 4-OHE₂, NAcCys,
4-OHE₂-2-NAcCys conjugates, and 4-OHE₂-1-N3Ade adduct
in the presence of 2E9 MAb.

| | Association | Association | Dissociation | |
|---|---|---|---|---|
| | constant, | rate, kₒₙ | rate, k_{off} | 1/k_{off} |
| Analytes | K_{A} (M⁻¹) | (M⁻¹ · sec⁻¹) | (sec⁻¹) | (sec) |
| 4-OHE₂-NAcCys^{a)} | 1.8 · 10⁸ | 4.6 · 10⁵ | 2.6 · 10⁻³ | 391 |
| 4-OHE₂-NAcCys- | 0.5 · 10⁸ | 2.7 · 10⁵ | 5.4 · 10⁻³ | 185 |
| SAMSA | | | | |
| 4-OHE₂ | 2.5 · 10⁵ | 1.5 · 10⁴ | 6.2 · 10⁻² | 17 |
| NAcCys | 2.1 · 10⁶ | 7.7 · 10⁴ | 3.7 · 10⁻² | 27 |
| 4-OHE₂-1-N3Ade^{b)} | 6.7 · 10⁴ | 6.5 · 10³ | 9.7 · 10⁻² | 10 |

^{a)}Similar values were obtained for 4-OHE₁-2-NAcCys;
^{b)}Similar values were obtained for 4-OHE₁-1-N3Ade.

Differences in affinity and reaction rates reported in Table I are immmediately apparent. For example, the value of K_{A} for the 2E9 MAb-4-OHE₂-2-NAcCys complex is very high (K_{A}=1.8·10⁸ M⁻¹), and is higher by a factor of 100 than that obtained for the NAcCys and about 2,700 times higher than K_{A} measured for the 4-OHE₂-1-N3Ade adduct. The short time of the equilibrium reaction between the 4-OHE₂-NAcCys and the 2E9 MAb allows for fast detection of 4-OHE₂-NAcCys. A high value of kn for the 4-OHE₂-NAcCys ensures efficient binding to MAb. The latter, along with a very low dissociation rate, allows for very efficient MAb -4-OHE₂-NAcCys complex formation, thus allowing for selective and sensitive detection of 4-OHE₂-NAcCys conjugates in human fluids, such as urine, serum; etc. The values of the parameters shown in Table I clearly indicate that 4-OHE₂-NAcCys can successfully compete with other closely related analytes for the binding sites of the MAb. For instance, the value of kₒₙ for the 2E9 MAb -4-OHE₂-2-NAcCys complexes is ˜30 times higher than that for 4-OHE₂ and ˜70 times higher than that for 4-OHE₂-1-N3Ade, thus leading to much slower binding of estradiol and 4-OHE₂-1-N3Ade by the 2E9 MAb. The dissociation time (1/k_{off}) is also very important, since short dissociation times for unwanted analytes (e.g., 4-OHE₂, NAcCys; etc.) allow their easy removal by a controlled washing procedure. For example, 1/k_{off} for 4-OHE₂-2-NAcCys is ˜6.5 min, which is more than one order of magnitude higher than 1 k_{off} for 4-OHE₂, NAcCys, and 4-OHE₂-1-N3Ade. Moreover, MAb specific for detection of 4-OHE₂-1-N3Ade adducts also can be used.

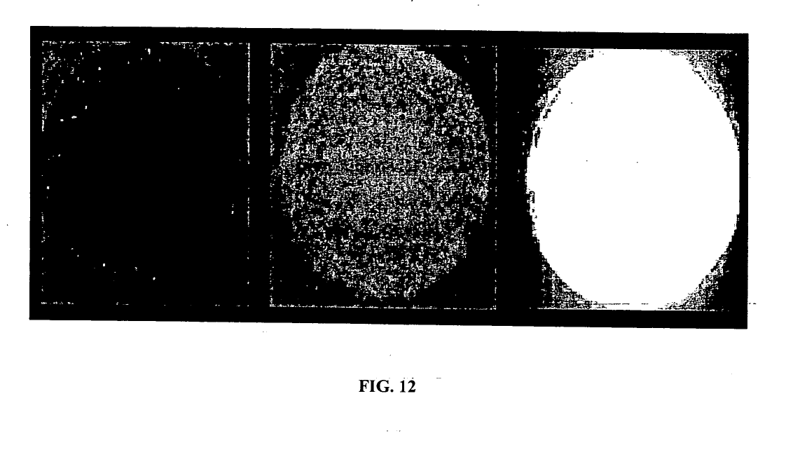

In one embodiment of the present invention, the biosensor active spots were prepared on a gold biosensor chip with suitable MAbs and were used to generate the preliminary data shown in FIG. 12. The active spot areas were ˜2.5 mm in diameter and were pre-activated with carbonyl diimidazole for efficient binding to MAbs (0.1 mg/mL, 1 μL/spot overnight, blocked with 1% BSA, 0.01% Triton-X100 in PBS for 2 hours at 37° C.). A hydrophobic barrier coating around the active spots significantly decreased the nonspecific binding. After MAbs were attached to the active spots of the biosensor, the chips were washed with PBS buffer containing Triton-X100 (0.01%). In order to estimate the capacity of a single spot on a chip, a calibration curve for the 4-OHE₂-NAcCys-SAMSA was generated. In order to accomplish this, spots were incubated (T=300 K) for 60 sec with 1 μL of 4-OHE₂-NAcCys-SAMSA at different concentrations and then washed. A linear dependence was observed in the range of about 10⁻¹⁶-10⁻¹⁰ moles. Saturation was observed at concentrations slightly higher than 10⁻¹⁰ moles.

To obtain the calibration curve for 4-OHE₂-NAcCys the spots on the chip were first incubated (for 10 min at 37° C.) with different concentrations of 4-OHE₂-NAcCys in a buffer solution. After washing for 30 sec, 1 μL of 10 μM 4-OHE₂-NAcCys-SAMSA solution was applied to each spot for 60 sec, and washed again for 30 sec. The K_{A} value of the reporter analyte is several times smaller than that for 4-OHE₂-2-NAcCys. Higher concentrations of 4-OHE₂-NAcCys-SAMSA and significantly longer exposure times to the reporter molecule led to measurable competition with 4-OHE₂-NAcCys. However, a short time exposure (60 sec) and a controlled washing procedure provided excellent and reproducible results with a linear calibration curve for 4-OHE₂-NAcCys, with the concentration ranging from 10 fmoles to 100 nmoles. Examples of three fluorescence-based images obtained for three different concentrations of 4-OHE₂-NAcCys are shown in FIG. 12. Fluorescence-based images from the left to the right correspond to a decreasing concentration of 4-OHE₂-2-NAcCys conjugate (c=10⁻¹⁵, 10⁻¹³, and 5×10⁻¹¹ moles). As expected, the fluorescence intensity increased from left to right, leading to brighter images, thus reflecting a concentration decrease of 4-OHE₂-2-NAcCys on the biosensor surface, as 4-OHE₂-2-NAcCys alone does not fluoresce. Thus, the more 4-OHE₂-2-NAcCys on the biosensor surface, the darker the image, in agreement with the “first-come-first-served” concept.

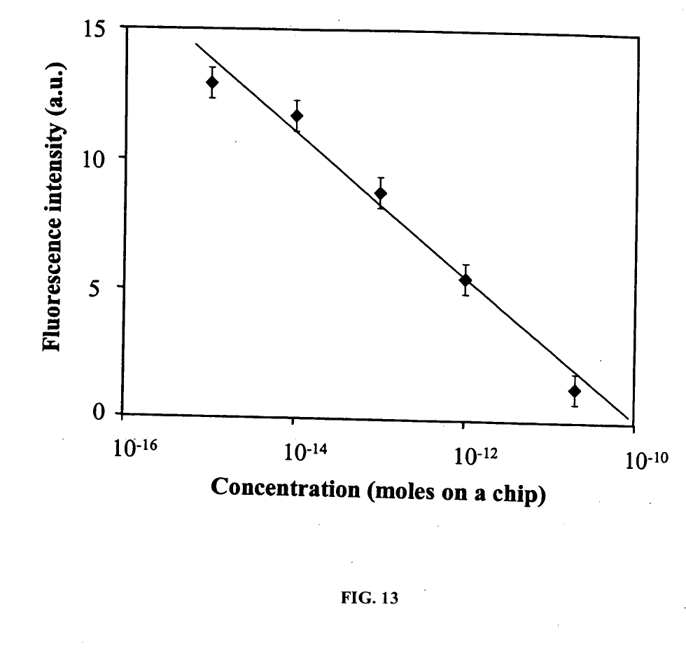

The calibration curve for 4-OHE₂-2-NAcCys, shown in FIG. 13, demonstrates that the femtomole detection limit is feasible. Similar data were obtained for transparent capillaries packed with MAb-dressed beads. This concept is described more fully below.

Preliminary results show that 2E9 MAb-based nanoassemblies are suitable for the detection and quantitation of 4-OHE₂-2-NAcCys conjugates. This methodology can serve as a breast and prostate cancer risk assessment.

An immunoaffinity biosensor column can be built and quantitative imaging capabilities with microsize beads and/or various microporous materials equipped with different MAbs can be used for selective biosensing processes and sensitive detection of analytes of interest. Alternatively, an MAb-based biosensor (biochip) can be constructed on a glass, polymer, and/or silicon wafer substrate with a single and/or multiple addressable surface patch(es) for sensitive and selective detection of estrogen-derived biomarkers. In both embodiments, detection can be based on the “first-come-first-served” approach and fluorescence-based imaging. As a first step towards developing such devices with room-temperature fluorescence-based imaging, the standards of the analytes of interest have to be derivatized with suitable fluorescent labels that will serve as reporter molecules. In one approach, the CEQ-derived conjugates and CEQ-DNA adducts are derivatized with SAMSA (or other fluorescent dye) via a specifically designed linker, while in another approach the analytes of interest are labeled with different sizes of quantum dots. MAb-based columns and/or biosensor chips designed for selective detection of analyte Aₓ are exposed to a complex fluid sample to extract selectively Aₓ. With established kinetics parameters for a specific MAb and analytes of interest, as well as a reporter molecule (i.e. K_{A}, kₒₙ and k_{off}), the columns and/or biochips are appropriately washed, ensuring that only a negligible amount of Aₓ, can be washed away. Subsequently, the column and/or biochip is exposed to a standard of Aₓ, labeled with a fluorescent dye and/or quantum dot (i.e. Aₓ*). After a second washing step (to remove un-complexed Aₓ*), a room-temperature fluorescence-based image is generated. The more Aₓ is retained on the column and/or biochip, the darker the image that is observed. As illustrated in FIG. 13, where a linear calibration curve for the detection of 4-OHE₂-2-NAcCys over several orders of magnitude is illustrated, this methodology can provide simple and efficient screening of estrogen-derived biomarkers in human fluids.

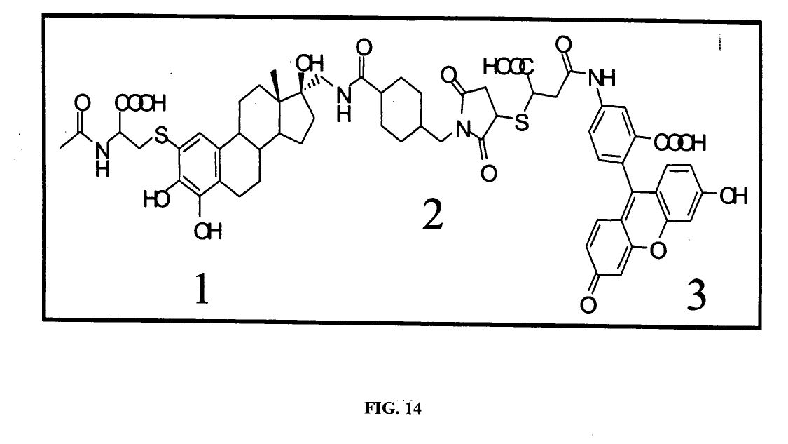

In order to ensure that the photostability of labeled analytes is extremely high, the analyte of interest can be derivatized with a selected fluorophore, for example SAMSA. The first step involves the removal of the acetyl group of SAMSA with a base like NaOH. After that, the hapten (6), (12) or (14) is reacted with it, such that the maleimide moiety of the hapten is connected with the free SH group of SAMSA as shown in FIG. 14.

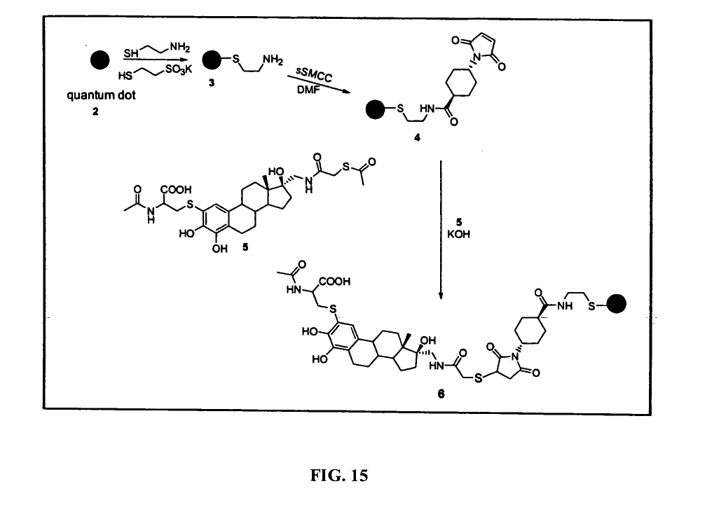

The analytes of interest can also be derivatized with differently sized quantum dots, which are known to possess exceptional stability, high quantum yield, broad absorption and narrow emission bands. Since the color of quantum dots emission depends on their size, and the emission spectra are very narrow, derivatization of analytes of interest with different sizes of nanocrystals allows detection of several biomarkers simultaneously. For example, ZnO-coated (TOPO-stabilized) quantum dots (FIG. 15, (2)) can be used. The first step involves the exchange of TOPO ligands for 2-aminoethanethiol/potassium 2-thioethanesulfonate to produce quantum dots with several amino groups (FIG. 15, (3)). All amino functional groups can be saturated using an excess of succinimidyl 4-(N-maleimido-methyl)cyclohexane-1-carboxylate (SMCC) to provide substrate (FIG. 15, (4)).

The final step involves Michael addition of the activated thiol group of the linker attached to 4-OHE₂-2-NAcCys (FIG. 15, (5)), for example, to the maleimide functionality of derivatized quantum dots (FIG. 15 (4)). A large excess of (FIG. 15, (4)) can lead to a 1:1 quantum dot conjugate—analyte of interest assembly (FIG. 15, (6)). Reaction mixtures can be passed through an affinity column, and unreacted quantum dots can be returned to the reaction cycle to achieve a higher conversion rate. After eight consecutive cycles, product is washed off the affinity column and used as reporter molecules in biosensor/column-based approaches. Strong and stable emission of quantum dots can increase imaging capabilities.

In view of the above, the present invention also provides a biochip comprising a monoclonal antibody having specificity for a conjugate and/or a DNA adduct derived from CEQ. Additionally, a kit comprising the biochip, a hapten, and a reporter molecule is provided.

### EXAMPLES

The following examples serve to illustrate the present invention. The examples are not intended to limit the scope of the invention in any way. Production and use of the antibodies, molecules, fluorescent markers, and other molecules and moities used in the following examples, as well as the expenmental methodologies discussed below, are either discussed above or are known in the art, as disclosed, for example, in Molecular Cloning: A Laboratory Manual, Joseph Sambrook, David W. Russell, Cold Spring Harbor Press (2001); High Throughput Screening: Methods and Protocols (Methods in Molecular Biology, 190), William P. Janzen, Humana Press (2002); Antibody Engineering: Methods and Protocols (Methods in Molecular Biology), Benny K. C. Lo, Human Press (2004); and Using Antibodies: A Laboratory Manual, Edward Harlow, David Lane, Cold Spring Habor Press (2001).

### Example 1

This example describes the synthesis of CEQ-derived conjugates.

4-OHE₁ and 4-OHE₂ were synthesized according to Dwivedy et al. (Chem. Res. Toxicol. 5, 828-833 (1992)). The 4-OHE₁- and 4-OHE₂-derived NAcCys conjugate standards were synthesized as previously described (Cao et al., Chem. Res. Toxicol. 11, 917-924 (1998)). The 4-OHE₁(E₂)-1-N7Gua (Stack et al., Chem. Res. Toxico. 9, 851-859 (1996)) and 4-OHE₁(E₂)-1-N3Ade (Li et al., Carcinogenesis 25, 289-297 (2004)) adducts were prepared in the Cavaliera/Rogan laboratory. Cys, NAcCys, and spectrophotometric grade ethanol were purchased from Aldrich (Milwaukee, Wis.). Ultra-pure grade glycerol was obtained from Spectrum Chemical (Gardena, Calif.). The high purity of standards of CEQ-derived conjugates, originally separated by HPLC, was verified by CE, which possesses higher separation power than HPLC. All CEQ-derived conjugates were kept for longer storage at −80° C. under an inert atmosphere (N₂ or Ar), since they are heat- and oxygen-sensitive. Special care was taken since the above conjugates are susceptible to oxidation in air in the presence of small amounts of cations to give disulfides (via a mercaptide). Therefore, samples were dissolved in methanol/buffer (80:20), with the following buffer content: 0.1 M CH₃COONH₄ and 1 mg/mL ascorbic acid in nanopure water, pH 4.5.

Analytical HPLC was conducted on a Waters 2695 Separations Module equipped with a Waters 996 photodiode array detector and a reversed phase Phenomenex Luna-(2) C-18 column (250×4.6 mm, 5 μm; 120 Å, Torrance, Calif.). Preparative HPLC was conducted on a Waters 600E solvent delivery system equipped with a 996 photodiode array detector and Phenomenex Luna-(2) C-18 column (300×21.2 mm, 10 μm; 120 Å, Torrance, Calif.). NMR spectra were recorded on a Varian Inova-500 instrument operating at 499.6 MHz and 125.62 MHz for ¹H and ¹³C, respectively, and referenced with deuterated solvents.

Fast atom bombardment tandem mass spectrometry (FAB-MS) was conducted at the Nebraska Center for Mass Spectrometry (University of Nebraska-Lincoln) using a MicroMass AutoSpec high resolution magnetic sector mass spectrometer (Manchester, England). Xenon was admitted to the collision cell at a level to attenuate the precursor ion signal by 75%. Data acquisition and processing were accomplished using OPUS software that was provided by the manufacturer (Microcasm). Samples were dissolved in 5-10 μL of methanol; 1 μL aliquots were placed on the sample probe tip along with 1 μL of a 1:1 mixture of glycerol/thioglycerol.

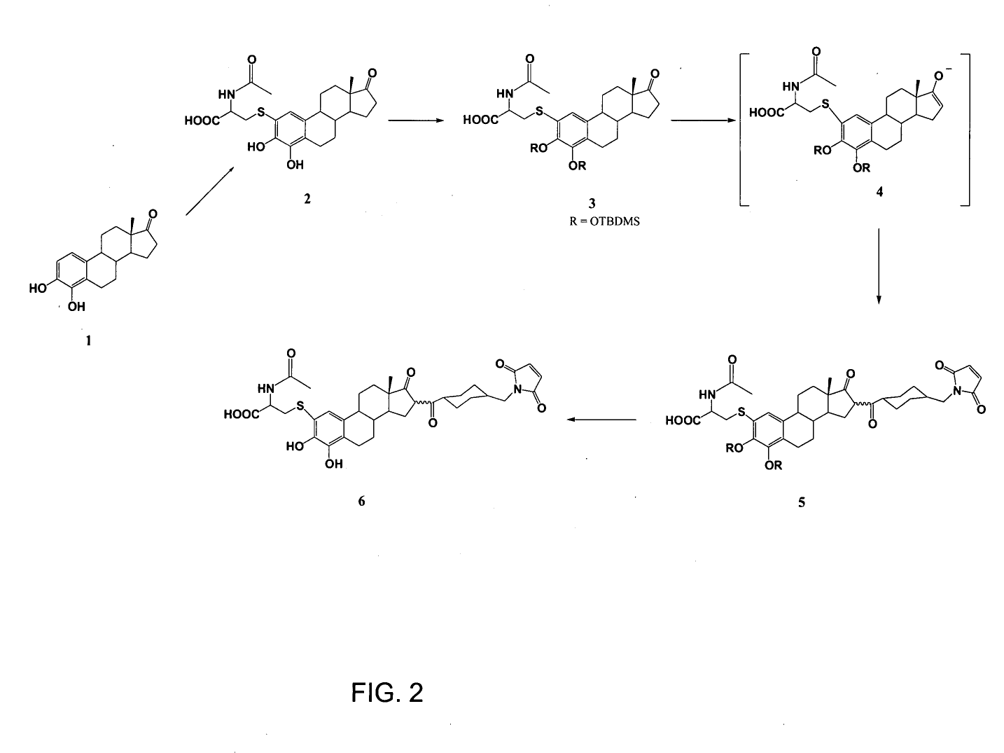

All reactions were performed using oven-dried glassware under an atmosphere of dry argon. Tert-butylchlorodimethylsilane (TBDMS-Cl), n-butyllithium, MnO₂, succinimidyl 4-(N-maleimidomethyl)-cyclohexanecarboxylate (SMCC), tetra-n-butylammonium fluoride, and anhydrous tetrahydrofuran (THF) were purchased from Aldrich Chemical Co. and used as such without further purification. 4-OHE₁ (1) was synthesized as described earlier (Dwivedy et al., Chem. Res. Toxicol. 5, 828-833 (1992)). Synthesis of the 4-OHE₁(E₂)-2-NAcCys-16a,β-MCC linker is summarized in FIG. 2. The conjugate was purified by reverse phase HPLC and its structure was verified by NMR.

To a stirred solution of 4-OHE₁ (100 mg, 0.35 mmol) in acetonitrile (10 mL) was added MnO₂ (200 mg) at 0° C. and stirred for 20 min. The yellowish green quinone was filtered directly into a stirred solution of NAcCys (116 mg, 0.70 mmol) in 6 mL of acetic acid/water (1:1, v/v). After 30 min, the reaction mixture was filtered and the product was separated on preparative HPLC, by starting with 10% acetonitrile/90% water (0.4% acetic acid) and increasing acetonitrile up to 100% linearly in 75 min at a flow rate of 6 ml/min to give 124 mg of (2) in ˜80% yield. UV: λₘₐₓ=289.4 nm. ¹H NMR (500 MHz, DMSO-d₆): 8.60 (s, 2H, Ar—OH, exchangeable with D₂O), 6.77 (s, 1H, 1-H), 4.42 (dd, J=4.4, 8.8 Hz, 1H, a-H-Cys), 3.14 (dd, J=4.4, 13.4 Hz, 1H, β-H-Cys), 2.93 (dd, J=8.8, 13.7 Hz, 1H β-H-Cys), 2.78 (dd, J=5.3, 17.4 Hz, 2H, 6-H), 2.60-0.90 (14H, remaining protons), 1.82 (s, 3H, CH₃CO), 0.75 (s, 3H, 13-CH₃). FAB-MS: m/z 448.1799 [(M+H)⁺, C₂₃H₃₀NO₆S, calc 448.1794].

To a stirred solution of 4-OHE₁-2-NAcCys (2) (100 mg, 0.22 mmol) in dry DMF (2 mL) was added TBDMS-Cl (2 mL, 1 M solution in CH₂Cl₂) under argon at room termperature. Dimethylaminopyridine (DMAP) (244 mg, 2 mmol) was added and the mixture was allowed to stir for 6 h. The product was analyzed on HPLC, by starting with 10% acetonitrile/90% water (0.25% TFA) for 10 min, followed by a linear gradient up to 100% acetonitrile in 25 min at a flow rate of 1 mL/min. The compound was purified on preparative HPLC by using initially 10% acetonitrile/90% H₂O (0.4% TFA) for 5 min, followed by a linear gradient up to 100% acetonitrile in 30 min at a flow rate of 7 mL/min. The compound 3 was eluted between 30-32 min, with a purified yield of 61.6 mg (50%). UV: λₘₐₓ32 289.4 nm. ¹H NMR (CDCl₃): 8.80 (bs, 2H, exchange with D₂O), 7.03 (s, 1H,1-H), 6.54 (d, J=7.0 Hz, 1H, NH exchange with D₂O), 4.76 (dd, J=6.5, 11.5 Hz, 1H, a-H-Cys), 3.29 (dd, J=4.0, 14.0 Hz, 1H, β-H-Cys), 3.13 (dd, J=6.5, 14.0 Hz, 1H, β-H-Cys), 2.93 (dd, J=3.5, 18 Hz, 1H, 6-H), 2.61 (m, 1H, 6-H), 2.52-1.25 (m, 13H, remaining protons), 1.97 (s, 3H, CH₃CO), 1.01 (s, 9H, 3×CH₃), 0.91 (s, 3H, 13-CH₃), 0.24/0.23 (s, 6H, 2×CH₃), FAB-MS: m/z 562.2601 [(M+H)⁺, C₂₉H₄₄NO₆SSi, calc. 562.2580].

4-O-TBDMS-E₁-2-NAcCys (3) (56.2 mg, 0.1 mmol) was dissolved in 2 mL of dry THF under argon and cooled to −78° C. Into this stirred solution was added slowly n-BuLi (250 μL, 2 M sol. in cyclohexane) via a cannula. The mixture was allowed to warm to room temperature and stirred for 30 min to produce the anolate (4). The temperature was lowered again to −78° C. and solid SMCC, (167.16 mg, 0.5 mmol) was added portion-wise under argon atmosphere. The mixture was stirred for 3 h and then quenched with 2 mL of Q saturated solution of NH₄Cl. THF was evaporated at low pressure and the solid residue was re-dissolved in DMF/CH₃OH (2 mL). The product was purified on preparative HPLC, by using initially 50% acetonitrile/50% water for 5 min and then increasing the proportion of acetonitrile linearly up to 100% in 25 min to afford 4-O-TBDMS-E₁-2-NAcCys-16a,β-MCC (4); yield 11.7 mg (15%). UV: λₘₐₓ=291.8 nm. ¹H NMR (DMSO-d₆): 8.13 (bs, 3H, exchangeable with D₂O), 6.99 (s, 2H, 2-H-maleimide, 3-H maleimide), 6.75 (s, 1H, 1-H), 4.19 (dd, J=4.4, 8.8 Hz, 1H, a-H-Cys), 3.10 (dd, J=4.4, 13.7 Hz, 1H, β-H-Cys), 2.90 (dd, J=8.8, 13.7 Hz, 1H β-H-Cys), 2.73 (dd, J=5.9, 18.1 Hz, 1H, 6-H), 2.62-0.6 (m, 25H, remaining protons), 1.70 (s, 3H, CH₃CO), 0.95 (s, 9H, 3×CH₃), 0.71 (s, 3H, 13-CH₃), 0.31 (s, 6H, 2×CH₃).

4-O-TBDMS-E₁-2-NAcCys-16a,β-MCC (5) (5 mg, 6.4 μmol) was dissolved in THF at 0° C. and tetrabutylammonium fluoride (1.5 eq) was added under argon. The reaction mixture was stirred at the same temperature for 30 min. The mixture was diluted with 5% HCl solution, and THF was evaporated at low pressure. The residue was dissolved in DMF/CH₃OH (2 mL), and filtered and purified on preparative HPLC by using 10% acetonitrile/90% water for 5 min, followed by a linear increase in acetonitrile concentration to 100% in 35 min at a flow rate of 5 mL/min. The peak of the required compound was eluted at a retention time of 24-26 min; yield 3.8 mg (89%). ¹H NMR (CDCl₃): 11.01 (s, 1H, exchangeable with D₂O), 8.27 (s, 1H, Ar—OH, exchangeable with D₂O), 8.25 (s, 1H, Ar—OH, exchangeable with D₂O), 6.97 (s, 2H, 2-H-maleimide, 3-H-maleimide), 6.75 (s, 1H, H-1), 6.60 (s, 1H, NH, exchange with D₂O, 4.20 (ddd, J=7.8, 4.9, 3.9 Hz, 1H, a-H-Cys), 3.40 (m, 2H), 3.25 (dd, J=13.7, 4.39 Hz, 1H, β-H-Cys), 3.13 (m, 1H, β-H-Cys), 2.82-0.85 (m, remaining 27 protons), 0.79 (s, 3H, 13-CH₃), FAB-MS: m/z 667.2675 [(M+H)⁺C₃₅H₄₃N₂O₉S, calc. 667.261 1].

The hapten for 4-OHE₂-2-NAcCys conjugate also can be made by the following efficient method: Synthesis of 3,4-isoproplylene (7): 4-OHE₁ (100 mg, 0.35 mmol), 2,2-dimethoxypropane (200 μl) and a catalytic amount of P₂O₅ were suspended in dry toluene. The mixture was heated under reflux with a soxhlet's extractor containing CaCl₂. The reaction mixture was refluxed for 2 h. Additional 2,2-dimethoxypropane was added if require for the completion of the reaction. Refluxing was continued until TLC showed no starting material. The reaction mixture was treated with 1 M solution of Na₂CO₃ (10 mL) after cooling to room temperature. The organic layer was separated, and the aqueous layer was extracted with hot toluene (2 times). Combined toluene layers were washed with water and dried over sodium sulfate. After evaporation of solvent, a dark yellow colored oil was obtained and purified on a silica gel column. Yield 70%. ¹H NMR (CDCl₃): 6.72 (d, J=8.3 Hz, 1H, H-1), 6.56 (d, J=8.3 Hz, 1H, H-2), 2.84 (dd, J=5.4, 8.5 Hz, 1H, H-6), 2.65 (m, 1H, H-6), 2.50 (dd, J=8.7, 16.1 Hz, 1H, H-16), 2.35 (m, 1H), 2.36 (m, 1H), 2.23-2.0 (m, 4H), 1.98-1.90 (m, 1H), 1.67 (s, 3H, CH₃), 1.66(s, 3H, CH₃), 0.91 (s, 3H, CH₃), 1.70-1.35 (m, remaining H). FAB-MS: m/z 327.4312 [(M+H)⁺] corresponding to C₂₁H₂₇O₃ calc. 327.4293.

Synthesis of protected cyanohydrine (8): Under argon and at room temperature a 25 mL round bottom flask was charged with anhydrous THF (0.5 mL), lithium methoxide (1.2 mg) and trimethylsilyl cyanide (250 ul). The resulting yellow-colored solution was stirred for 10 min, and solid 3,4-isoproplylene (7, 172 mg, 0.53 mmol) was added. The stirring was continued for 6h. After completion, the reaction was quenched with 10% Na₂CO₃ (3 mL) and extracted with tert-butyl methyl ether (3 times). Combined ether layers were evaporated to afford an oily product and used as such for the next step. Yield 225 mg (95%).

Synthesis of aminomethylestradiol (9). The crude 8 (225 mg) was dissolved in toluene (2 mL), and 300 ul of RedAl® were added. The reaction mixture was stirred at 70° C. for 4 h and then at room temperature overnight. Completion of reaction was checked by TLC. The reaction was carefully quenched with 1 M NaOH (2 mL) and the resulting two layers were shaken and let to separate. The upper layer (organic) was removed; the aqueous layer (lower) was extracted with hot toluene (2 times) and combined. After evaporation, the gummy material was obtained and used as such for the next step. Yield 159 mg (˜70%).

Synthesis of 4-hydroxy-17-aminomethylestradiol (10). The crude 9 was treated with trifluoroactic acid at 100° C. for 5 min and then brought to room temperature. The mixture was let to stir at room temperature till HPLC analysis indicated the complete removal of the protective acetonoid group. After completion, the reaction mixture was directly injected to preparative HPLC under reverse phase condition to purify the target catechol (10). Yield 85%. ¹H NMR (DMSO-d₆): 7.80 (br s, 3H, exchangeable with D20 shaking), 6.66 (d, J=8.3 Hz, 1H, H-1), 6.55 (d, J=8.3 Hz. 1H, H-2), 2.93 (m, 1H, 17-CH₂NH₂), 2.88 (m, 1H, 17-CH₂NH₂), 2.73 (m, 1H), 2.66 (m, 1H), 2.50 (m, 2H), 0.82 (s, 3H, CH3), 2.20-0.9 (m, remaining H). FAB-MS: m/z 318.4356 [(M+H)⁺] corresponding to C₁₉H₂₈O₃ calc. 318.4306.

Synthesis of 4-hydroxy-17-aminomethlyestradiol-2-NacCys (11). The NAcCys-conjugate (11) was synthesized from 10 as described before. MS: m/z 479.6 [(M+H)⁺] corresponding to C₂₄H₃₄N₂O₆S.

Synthesis of 4-hydroxy-17-aminomethylestradiol-2-NacCys-MCC hapten (12). To a stirred solution of 11 (1 mg) in dry THF (1 mL) containing a few drops of Diisopropylethylamine (DIEA) 7 mg of solid SMCC was added. The reaction mixture was stirred at room temperature for 4 hr and then subjected to preparative HPLC for purification of hapten 12 under reverse phase condition. MS: m/z 698.8 [(M+H)⁺] corresponding to C₃₆H₄₇N₃O₉S.

Synthesis of 4-hydroxy-17-aminomethylestradiol-1-N3Ade (13). The catechol 10 (10 mg) was oxidized to quinone with MnO₂ (20 mg) in acetonitrile (2 mL) at 0° C. After 30 min, the yellowish green quinone solution was added to a stirred solution of adenine (10 eq) in mixture (1:1) of acetic acid/water. The reaction was stirred for 10 h, and then adenine adduct was purified by preparative HPLC.

Synthesis of 4-hydroxy-17-aminomethylestradiol-1-N3Ade-MCC (14). The hapten 14 was synthesized by using the conditions described for NAcCys-conjugate hapten (12). MS: m/z 670.7 [(M+H)⁺] corresponding to C₃₆H₄₃N₇O₆.

There is no immunological cross-reactivity between KLH and OA. Hence, positive hybridoma cell lines secreting antibody against 4-OHE₁(E₂)-2-NAcCys could be rapidly identified using OA-4-OHE₁(E₂)-2-NAcCys. An affinity column was developed and used to purify MAb against 4-OHE₁(E₂)-2-NAcCys. The purified MAb was immobilized on an agarose bead column. This column was used to capture and preconcentrate the hapten of interest out of urine samples. A number of structurally related standards were used to estimate the selectivity and specificity of chosen MAb. CE with field amplified sample stacking (FASS) in absorbance detection mode, and laser induced low temperature luminescence measurements were used to identify and quantitate the 4-OHE₁(E₂)-2-NAcCys conjugates and related compounds released from the affinity column. Femtomole detection limits have been demonstrated.

### Example 2

This example describes the production and screening of mouse hybridomas and MAbs.

OA and KLH were purchased from Pierce Biotechnology, Inc., Rockford, Ill. Delbecco's Modified Eagle medium and horse serum were purchased from Mediatech, Inc., Herndon, Va., and Valley Biomedical, Inc., Winchester, Va., respectively. N-(9-Fluorenyl)methoxycarbonyl multiple antigenic peptides (Fmoc MAP) resin was purchased from Applied Biosystems, Foster City, Calif. Well-established methods (Antibodies: A Laboratory Manual (E. Harlow et al.) Chapter 6, pp. 139-243, Cold Spring Harbor, 198829) were used to generate an immune response in the mice. The 4-OHE₁(E₂)-2-NAcCys-16a,β-MCC linker was conjugated to KLH and used in an immunization protocol with 25 μg of antigen/mouse/injection using Freund's incomplete adjuvant. Serum titers were established using 4-OHE₁(E₂)-2-NAcCys conjugated to OA. The NAcCys conjugate is the hydrolytic product of the corresponding conjugate with GSH [4-OHE₁(E₂)-2-Cys] followed by N-acetylation of cysteine. The mice were tested for an immune response to the 4-OHE₁(E₂)-2-NAcCys using OA-[4-OHE₁(E₂)-2-NAcCys] and OA alone. Mice demonstrated an elevated antibody titer with OA-[4-OHE₁(E₂)-2-NAcCys] compared to OA alone. The mouse with the highest titer was IP boosted with KLH-4-OHE₁-NAcCys and used for hybridoma production. Mouse spleen cells were fused with an equal number of SP2/O cells (40 million of each) and plated in 16×96-well microtiter plates. When hybridoma wells started to turn yellow, the plates were screened by ELISA using OA-4-OHE₁(E₂)-2-NAcCys OA as an antigen to immobilize captured MAb. Five hundred ng of OA-4-OHE₁(E₂)-2-NAcCys-16a,β-MCC in binding buffer (100 mM NaHCO₃, pH 9.3) were used to coat each well of a Nunc maxisorb plate. Most of the wells had optical density (OD) values of less than 0.1. However, the wells that had hybridomas secreting antibody to the 4-OHE₁(E₂)-2-NAcCys hapten were quite apparent. That is, the wells where the antibody was produced had much higher OD values, typically in the range of 0.5 to 1.0, clearly indicating that antibody was produced against the 4-OHE₁(E₂)-2-NAcCys conjugates.

### Example 3

This example describes the preparation of immunoaffinity columns.

An affinity column was made to purify MAb by immobilizing the 4-OHE₁(E₂)-2-NAcCys-16a,β-MCC on a MAP resin core used to commonly synthesize peptides. The hapten was immobilized on the MAP resin bead column using the same chemistry used to attach it to the carrier proteins (Jue et al. Biochemistry 17, 5399-5405 (1978)). This column was used to purify antibody by passage of 3 mL of supernatant fluid from the selected hybridoma over the column. The column was washed with 50 mL of PBS, and antibody was eluted with 100 mM acetic acid, pH 2.5. The eluted antibody (from hybridoma 2E₉) was isotyped using an isotyping kit specific for mouse antibody, confirming that the antibody was of mouse origin (IgG_{2b}κ) and not from the horse serum used to grow the cells. This purified MAb was immobilized on an agarose bead column (Aminolink kit, Pierce Inc.) and used to detect 4-OHE₁-2-NAcCys in PBS buffer that was spiked with various concentrations of the conjugate.

### Example 4

This example describes competitive ELISA for 4-OHE₁-2-NAcCys.

Wells were coated with OA-40HE₁(E₂)-2-NAcCys-16a,β-MCC, 500 ng/mL, 50 μL/well overnight, and blocked with 1% non-fat dried milk, 0.01% Triton-X100 in PBS for 2 h at 37° C. Competition curves were generated as described below. MAb and competitor molecules 4-OHE₁(E₂)-2-NAcCys (1), NAcCys (2), 4-OHE₁(E₂) (3), and 4-OHE₁(E₂)-1-N3Ade (4) were added to the wells at the same time and allowed to react with immobilized antigen for one hour before secondary antibody was added. The plates were washed with PBS with Triton-X100 (0.01%). Secondary antibody labeled with horseradish peroxidase (HRP) was used according to manufacturer's (BRL, Inc.) recommendations and incubated at 37° C. for 1 h. Plates were washed again, 2,2′-azino-bis(3-ethylbenzthiazoline-6-sulfonic acid) (ABTS) was added, and the plates were read at 405 nm on a BioRad-EL800 plate reader. The specificity of the 2E₉ MAb raised against 4-OHE₁(E₂)-2-NAcCys was confirmed, and cross-reactivity to a number of related compounds was established.

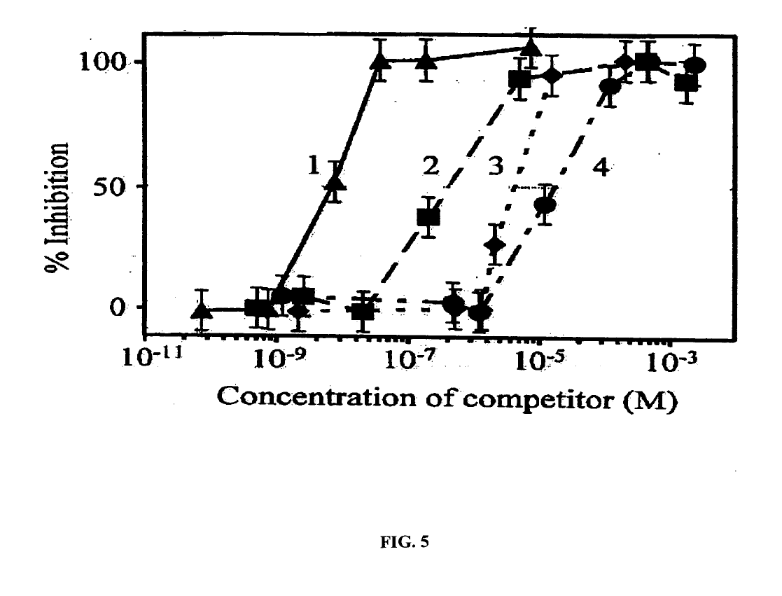

The results are shown in FIG. 5, where the competitor-mediated reduction of MAb binding is expressed as % inhibition versus untreated MAb and plotted as a function of the concentration of competitor in the wells of the ELISA plate. Inhibition curves were developed for several analytes of interest; the I₅₀'s (quantity producing 50% inhibition of MAb binding in the ELISA) of these analytes were determined by regression analysis. Comparison of curves 1-4 of FIG. 5 reveals that, in addition to high affinity binding of the 4-OHE₁-2-NAcCys to the 2E₉ MAb, the latter discnminates very well the analyte of interest from closely related analytes like the 40HE-₁-N3Ade adduct, NAcCys, 4-OHE₁ and 4-OHE₂. The significantly reduced binding for 4-OHE¹ and 4-OHE-₁-N3Ade suggests that hydrogen bonding may play a central role in the MAb-4-OHE₁(E-₂)-2-NAcCys complex formation. For example, the I₅₀ of 4-OHE-₁-N3Ade (I₅₀=1.5·10⁻⁵M) was about 2700 times higher than the I₅₀ of the 4-OHE₁-2-NAcCys (I₅₀=5.6·10⁻⁹M).

### Example 5

This example describes the use of indirect ELISA to measure the association/dissociation rate constants.

An ELISA was used to measure the association/dissociation rate constants of MAb-hapten (i.e., MAb-4-OHE₁-2-NAcCys) and/or association/dissociation rate constants of related analytes. The MAb and suitable hapten(s) were mixed in solution to initiate the equilibrium reaction. At different time intervals, the amount of the free MAb in the reaction mixture was determined by an indirect ELISA. The association rate constants for 4-OHE₁-2-NAcCys, 4-OHE₁, NAcCys, and 4-OHE₁-1-N3Ade were estimated by nonlinear regression against an equation introduced from the derivation of the mass balance of MAb-antigen/related analyte interaction (Zhuang et al., J Biosci. Bioeng. 92, 330-336 (2001); Foote et al., Proc. Natl. Acad. Sci. USA 92, 1254-1256 (1995); Goldbaum et al., J Immunol. 162, 6040-6045 (1999); Northrup et al., Proc. Natl. Acad. Sci. USA 89, 3338-3342 (1992)).

To determine the bound fraction of the antibody by ELISA, 3 rows and 8 columns of the microtiter plate were incubated with sufficient concentration of antigen to saturate the wells. After washing the plate with PBS 3 times, 4 rows of the plate including 3 rows coated with the antigen were blocked with 1% non-fat dried milk, 0.01% Triton-X100 in PBS for 3 hrs. Then the plate was washed with PBS with Triton-X100 (0.01%) 3 times. Equal volumes (160 μL) of the antigen (or related analyte) and PBS were mixed, and the mixture was put into the first well of the row that was blocked but not coated with the antigen (non-coated row).

The average absorbance of this column (A_{b}) measured in the last step of the ELISA corresponds to the blank. Equal volumes (160 μL) of the antibody solution and PBS were mixed, and the mixture was put into the last (8th) well of the non-coated row. The average absorbance of this column (Aₒ) corresponds to the total concentration of the antibody. Equal volumes of the antibody solution and antigen (or related analyte) solution were mixed quickly, and the mixture was immediately put into the second well of the non-coated row. At appropriate time intervals (after 10, 20, 23, 26 and 28 min), a new mixture of the antibody and hapten (or related analyte) solution were mixed quickly, immediately put into the wells of the non-coated row from the 3rd to 7th well. The average absorbance of these columns (Aₜ) corresponds to the concentration of the free antibody at t=30, 20, 10, 7, 4 and 2 min for the 3rd to 8th well, respectively. Two min after the 7th well was filled with the mixture, using an eight-channel Pipetman, the mixtures on the row were quickly dispensed into the 3 rows that were coated with the antigen (or related analyte, 50 μL each). After 1 min the mixture was discarded and the plate was washed with PBS with Triton-X100 (0.01%) 3 times. The secondary antibody solution (HRP-conjugated) was added to the plate (50 μL), and the plate was incubated at 37° C. for 1 h. After washing with PBS and Triton-X100 (0.01%) 3 times, the substrate was added to the plate (50 μL). After incubation for 10 min at room temperature, the absorbance at 405 nm was measured.

At equilibrium conditions for the MAb-hapten reaction, the affinity constant (K_{A}) for the binding of 2E₉ MAb to 4-OHE1-N3Ade was determined to be 6.7·10⁴ M-⁻¹ or 2700 times smaller than that to the 4-OHE₁-2-NAcCys (K_{A}=1.8·10⁸ M⁻). Association constants K_{A} for 4-OHE₁ and NAcCys were also determined and are 2.5·10⁵ and 2.1·10⁶ M⁻¹, respectively. The latter indicates that the K_{A} values for 4-OHE₁ and NAcCys are about 720 and 86 times smaller than the K_{A} obtained for the 4-OHE₁-2-NAcCys. Thus, as expected, the competition curves demonstrated high specificity for the 4-OHE₁-2-NAcCys conjugate in comparison with other structurally similar compounds, as summarized in Table I. In addition to the values of K_{A}, the association/dissociation rate constants (kₒₙ/k_{off}) are important parameters for characterizing MAb-hapten interactions. When the time required for the equilibrium reaction between the antigen and the antibody is shorter, one can detect or quantify the hapten faster. The binding fraction of MAb at time t, is given by Fₜ, (i.e., the ratio of bound MAb to total amount of MAb) and is often written as:

$${F}_{t} = \frac{{A}_{t} - {A}_{b}}{{A}_{0} - {A}_{b}} = \frac{1 - exp [ - {k}_{on} ( {Ag}_{tot} + {K}_{d} ) t ]}{1 + {K}_{d} / {Ag}_{tot}}$$

where Aₜ, A_{b}, and A₀ correspond to the absorbance of ABTS at time ₜ, absorbance in the absence of MAb, and absorbance in the absence of antigen, respectively (Zhuang et al., J Biosci. Bioeng. 92, 330-336 (2001)). The kₒₙ is an association rate constant, Agₜₒₜ is the total concentration of hapten (or related analyte), and finally K_{d}=k_{off}/kₒₙ corresponds to the dissociation constant. The concentrations for 4-OHE₁(E₂)-2-NAcCys, 4-OHE₁, NAcCys, and 4-OHE₁-1-N3Ade of 7.1·10⁻⁷, 2.0−10⁻⁵, 5.84·10⁻⁶, and 2.9·10⁻⁵ M, respectively, were used to get at least four initial points at an unsaturated level on the binding fraction curve against the incubation time. Different concentrations of analytes were used, as the smaller the association constant between a particular analyte and MAb, the higher the necessary concentration of an analyte.

The equilibrium association constants (K_{A}) and association (kₒₙ)/dissociation (k_{off}) rates for 4-OHE₁(E₂)-2-NAcCys conjugates are given in Table I, which also provides information on other closely related analytes, such as 4-OHE₁(E₂), NAcCys, and 4-OHE₁(E₂)-1-N3 Ade adduct. All values listed in Table I have been determined using an indirect competitive ELISA (Antibodies: A Laboratory Manual (E. Harlow et al.) Chapter 6, pp. 139-243, Cold Spring Harbor, 1988). Differences in affinity and reaction rates reported in Table I are immediately apparent. For example, the value of K_{A} for the 2E₉ MAb-4-OHE₁(E₂)-2-NAcCys complex is very high (K_{A}=1.8·10⁸ M⁻¹), and is higher by a factor of 100 than that obtained for the NAcCys and about 2,700 times higher than K_{A} measured for the 4-OHE₂-1-N3Ade adduct. The short time of the equilibrium reaction between the 4-OHE₁(E₂)-2-NAcCys and the 2E₉ MAb allows for fast detection of 4-OHE₁(E₂)-2-NAcCys. A high value of the kₒₙ for the 4-OHE₁(E₂)-2-NAcCys ensures an efficient binding to MAb. The latter, along with a very low dissociation rate, allows very efficient MAb-4-OHE₁(E₂)-2-NAcCys complex formation, thus allowing selective and sensitive detection of 4-OHE₁(E₂)-2-NAcCys conjugates in human fluids such as urine, serum, etc. The values of parameters shown in Table I clearly indicate that 4-OHE₁(E₂)-2-NAcCys can successfully compete with other closely related analytes for the binding sites of the MAb. For instance, the value of the kₒₙ for the 2E9 MAb-4-OHE₁(E₂)-2-NAcCys complex is ˜30 times higher than that for 4-OHE₂ and ˜70 times higher than that for 4-OHE₂-1-N3Ade, thus leading to much slower binding of 4-OHE₁(E₂) and 4-OHE₁(E₂)-1-N3Ade by the 2E₉ MAb. The dissociation time (1/k_{off}) is also very important, since short dissociation times for unwanted analytes (e.g. 4-OHE₂-2-NAcCys, etc) allow their easy removal by a time-controlled washing procedure. For example, 1/k_{off} obtained for 4-OHE₂-2-NAcCys is ˜6.5 min, which is more than one order of magnitude higher than 1/k_{off} for 4-OHE₂, NAcCys, and 4-OHE₂-1-N3Ade. The MAb against 4-OHE₂-1-N3Ade (with K_{A}=2·10⁸ M⁻¹) have also been developed.

### Example 6

This example describes preparation of a human urine sample.

A clean-catch urine sample was collected from one healthy volunteer with no history of breast or prostate cancer. The sample was stored in aliquots at −80° C. Urine samples were filtered through a 0.22 μm filter (8110 μStar, Coming, Inc., Coming, N.Y.) and diluted 10-fold with PBS buffer. Urine samples were thawed and spiked with various known amounts of 4-OHE₁-2-NAcCys and passed over an agarose affinity column.

### Example 7

This example describes the use of CE to evaluate the binding affinities of the 2E₉ MAb developed for the detection of 4-OHE₁(E₂)-2-NAcCys conjugates.

CE was used to analyze a water-based buffer sample spiked with five analytes of interest: 1) 4-OHE₁-1-N3Ade, 2) 4-OHE₁, 3) 4-OHE₂, 4) 4-OHE₁-2-NAcCys, and 5 ) NAcCys. The concentration of analytes 1, 2, 3 and 5 used for the CE separation was about 10⁻⁶ M, while the concentration of the key analyte of interest was smaller by a factor of 100, i.e., 10⁻⁸ M. The analysis of samples was done with a P/ACE/MDQ CE system (Beckman Coulter, Fullerton, Calif.) with a photodiode array (PDA) detector for simultaneous detection of electropherograms and UV absorption spectra of separated analytes. A bare fused-silica capillary (Polymicro Technologies, Phoenix, Ariz.) with 30 cm effective length and 40.2 cm total length (75 mm i.d. and 360 mm o.d.) was used. The running buffer was 0.5% SDS surfactant in 25 mM Tris (pH 3.3 adjusted by H₃PO₄). The FASS method was used for analyte preconcentration. To achieve reproducible and accurate stacking results, a water plug was injected into the capillary before the sample at 0.2 psi for 0.2 min, then the sample was injected at −10 kV for 30 sec.

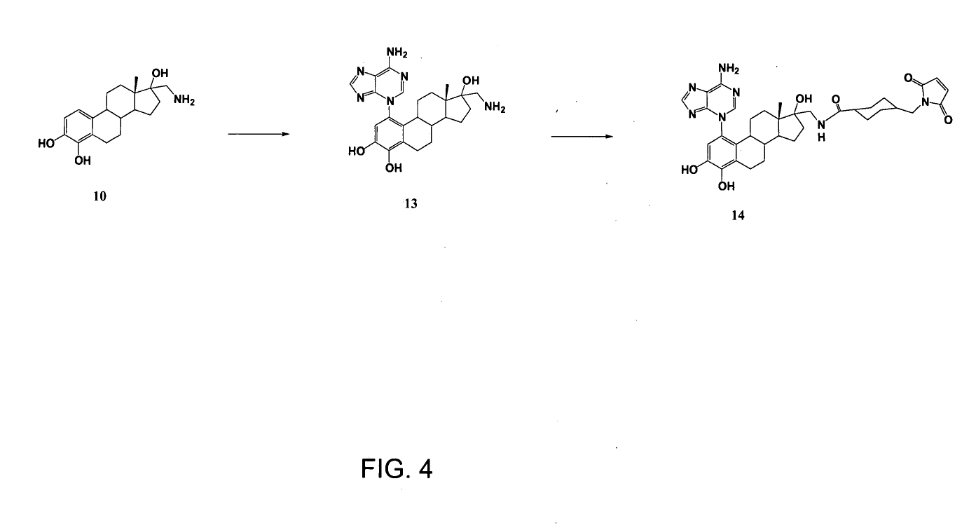

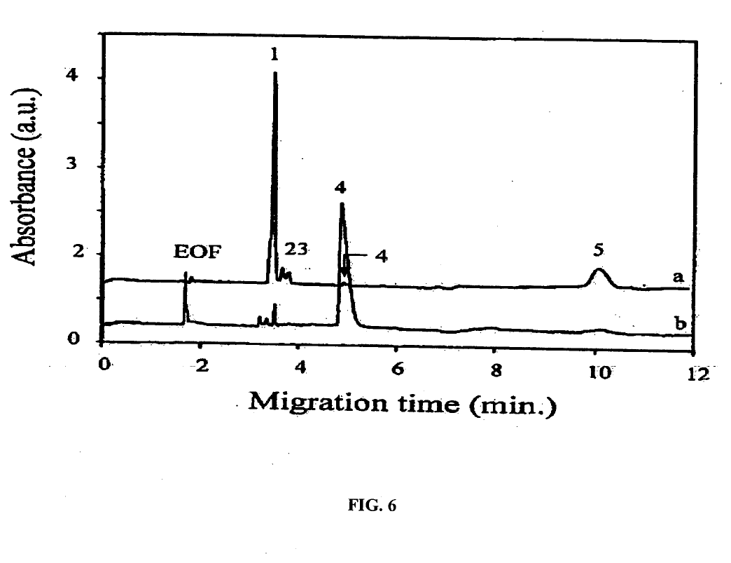

The results are shown in FIG. 6. The corresponding room temperature CE absorbance-based electropherogram (λ_{obs}=214 nm) is shown in FIG. 4 as curve a. The solid arrow in FIG. 6 indicates the position of analyte 4 as confirmed by standard spiking procedure with a higher concentration of 4-OHE₁-2-NAcCys. As expected, the peak corresponding to this analyte is hardly discernible in curve a of FIG. 4 (due to concentration differences), with the varying intensities of the remaining peaks due to large differences in the absorption coefficients at 214 nm. A specially prepared 2E₉ MAb-based affinity column was used to capture and concentrate the 4 OHE₁-2-NAcCys out of the above solution. The identify the analytes capture by the affinity column, the following procedure was utilized: first, about 2 mL of the sample was passed through the affinity column and after 10 min of incubation time, 20 mL of binding buffer was run over the column to wash out unbound analytes; second, the analytes captured by the column were released with the elution buffer (2 μL); third, the eluted sample was evaporated to dryness and re-dissolved in 20 μL of methanol leading to sample preconcentration by a factor of ˜100; and fourth, methanol was exchanged with a suitable buffer for subsequent CE separation. The resulting electropherogram, shown as curve b in FIG. 6, shows that mostly one analyte has been pre-concentrated by the affinity column. This peak (near 5 min) corresponds to the 4-OHE₁-2-NAcCys conjugate, as confirmed by standard spiking procedures and phosphorescence spectroscopy. The very small peaks near 3.5 and 10 min most likely correspond to peaks 1-3 and 5, respectively; however, only the identity of peak 5 was confirmed by the spiking procedure. The concentration of peaks 1-3 was too small for positive identification even when the sample was further concentrated by a factor of 10. Since the sample corresponding to electropherogram b (FIG. 6) was pre-concentrated by two orders of magnitude, we conclude that the binding efficiency for analytes 1, 2, 3 and 5 is negligibly small. Comparison of the integrated peak intensities in the electropherograms a and b reveals that the column preferentially captures the 4-OHE₁-2-NAcCys conjugate.

### Example 8

This example describes luminescence and absorption spectroscopy.

Luminescence spectra were obtained using an excitation wavelength of 257 nm of a Lexel 95-SHG-257 CW laser. Emission was dispersed by a Model 218 0.3-m monochromator (McPherson, Acton, Mass.), equipped with a 300 G/mm grating, providing a resolution of ˜1 nm, and a spectral window of approximately 200 nm. Spectra were detected with an intensified CCD camera (Princeton Instruments, Trenton, N.J.) using gated and non-gated modes of detection. A fast shutter, operated by a Uniblitz driver control (model SD-12-2B), was synchronized with the CCD camera (ICCD-1024 MLDG-E1) and used for time-resolved phosphorescence measurements. Using this setup, time-resolved phosphorescence spectra (˜10⁻⁴-10⁻³ M analyte concentrations) could be measured in 0.5 sec intervals with a gate width of 0.5 sec.

To ensure good glass formation, glycerol (50% by volume) was added to the samples in buffer just prior to cooling to 77 K in a liquid nitrogen optical cryostat with suprasil optical windows. Samples (ca. 20 μL) were contained in suprasil tubes (2-modified i.d.). Luminescence spectra of 4-OHE₁(E₂) and 4-OHE₁(E₂)-derived-NAcCys standards and samples released from the affinity column conjugates were measured at 77 K; all spectra were corrected for background luminescence.

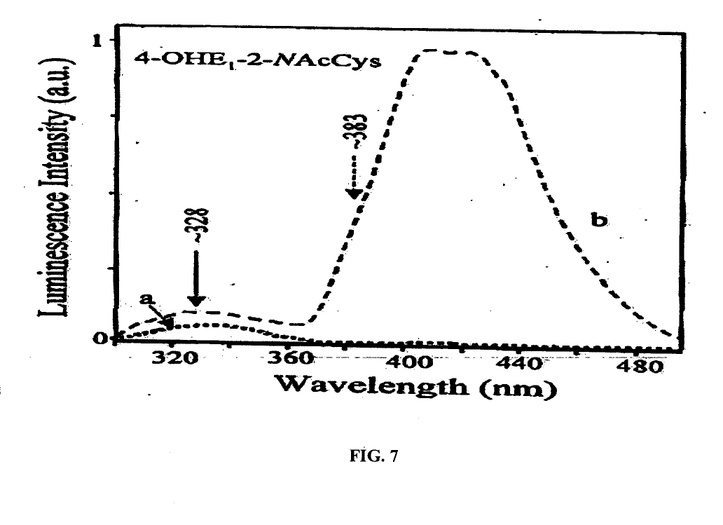

As a final test to prove that peak 4 in curve b corresponds to the 4-OHE₁-2-NAcCys conjugate, the elution extract was also analyzed by low temperature phosphorescence (77 K) spectroscopy. Off-line luminescence detection was used, as: 1) the concentration of the remaining analytes was negligibly small; 2) 4-OHE₁ and 4-OHE₂ are not phosphorescent at 77 K (Jankowiak et al., Chem. Res. Toxicol. 16, 304-311 (2003)); and 3) the 77 K phosphorescence spectra of NAcCys and 4-OHE-1-N3Ade are easily distinguishable from the phosphorescence spectrum of the 4-OHE₁-2-NAcCys conjugates (Jankowiak et al., Chem. Res. Toxicol. 16, 304-311 (2003); Markushin et al., Chem. Res. Toxicol. 16, 1107-1117 (2003)). The luminescence spectrum obtained for peak 4 is shown in FIG. 7. We recall that the 4-OHE₁-2-NAcCys is a breakdown product of the 4-OHE₁-2-SG (if present in urine) and could serve as a biomarker of exposure to CEQ. The emission spectrum shown in FIG. 5 was obtained at 77 K with an excitation wavelength of 257 nm in Gly/H₂O (pH 3). This luminescence spectrum (with its fluorescence origin band near 328 nm and very weak phosphorescence origin band near 383 nm) is in perfect agreement with our previous studies (Jankowiak et al., Chem. Res. Toxicol. 16, 304-311 (2003)) and corresponds to the 4-OHE₁-2-NAcCys; a strong phosphorescence is observed, as the lowest singlet and triplet states of 4-OHE₁-2-NAcCys are of n,π* and π,π* character, respectively (Jankowiak et al., Chem. Res. Toxicol. 16, 304-311 (2003)). Thus, luminescence spectroscopy can be used for identification of the 4-OHE₁-2-NAcCys and/or 4-OHE₂-2-NAcCys conjugates eluted from the 2E₉ MAb-based affinity column.

### Example 9

This example described competitive purification of 4-OHE₁-2-NAcCys from an agarose affinity column.

In order to show the utility and efficiency of affinity columns, a column with the 2E₉ MAb immobilized on agarose beads was made, washed with 20 mL of PBS buffer, and then adjusted with 10 mL of binding buffer. A urine sample from a healthy volunteer was diluted 10 times in PBS buffer. Then, 2 mL of diluted urine sample spiked with 20 μL of a 5 mM solution of 4-OHE₁-2-NAcCys was added to the column. After 10 min of incubation time, 20 mL of binding buffer was run over the column to wash out unbound analytes. After that , 2 mL of releasing buffer was added and the liquid was collected in 500 μL aliquots. In the next step the collected fractions were evaporated and redissolved in 20 μL of methanol for further low-T phosphorescence studies and/or CE experiments. Results from the phosphorescence experiment are shown in FIG. 6. The bars correspond to the integrated phosphorescence intensity obtained for the above-mentioned aliquots eluted from the affinity column. Data shown in frame A were obtained for 1 mL of buffer-diluted human urine sample spiked with 4-OHE₁-2-NAcCys at a concentration of 4×10⁻⁷ M. Data shown in frame B were obtained for the same sample diluted with PBS buffer by an additional factor of 100. Note that nearly the same amount of 4-OHE₁-2-NAcCys was recovered. Thus, the data shown in frames A and B demonstrate that although the concentration of 4-OHE₁-2-NAcCys in the above two experiments differed by two orders of magnitude, the combined efficiency of analyte recovery in the three runs was ˜80%, as estimated by the integrated phosphorescence intensity. The purity of all fractions was confirmed by CE. This indicates that the contribution from nonspecific binding is negligible, and excellent recovery of the analyte of interest from urine/buffer samples can be accomplished.

### Example 10

This example describes CE with FASS and absorbance/phosphorescence detection in spiked urine samples.

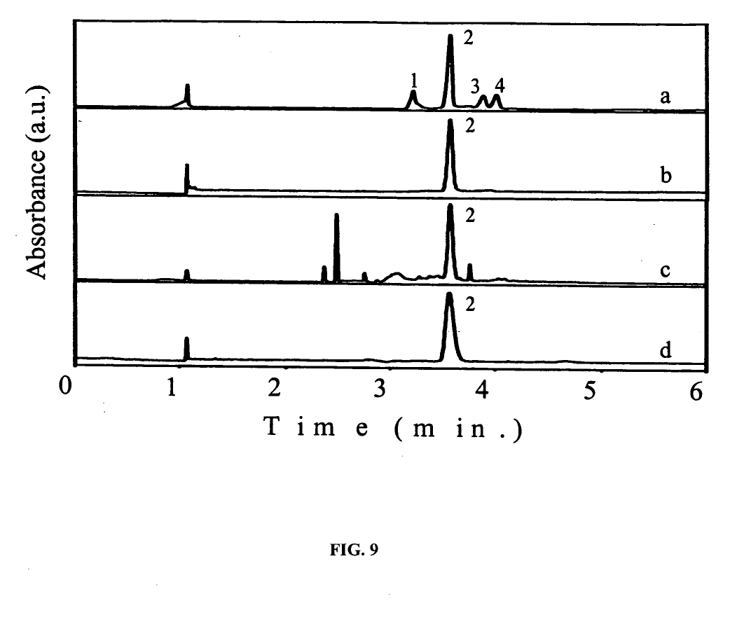

FIG. 9 shows four absorbance-based CE electropherograms to further demonstrate the selectivity of the MAb raised against one of the analytes of interest, i.e. 4-OHE₁(E₂)-2-NAcCys. Spectrum (a) corresponds to a CE electropherogram obtained for a mixture of 4-OHE₁-1-N3Ade (peak 1; c=5×10⁻⁵ M), 4-OHE₁-2-NAcCys (peak 2; c=10⁻⁴ M), 4-OHE₁ (peak 3; c=5×10⁻⁵ M), and 4-OHE₁₋1-N7Gua (peak 4; c=10⁻⁵ M) in a buffer solution. Curve (b) is the electropherogram of PBS buffer (2 mL) spiked with the mixture of the above four analytes diluted by a factor of 100 and run through the 2E₉ MAb-based affinity column. Only peak 2 is observed, with an ˜80% efficiency of recovery. Two orders of magnitude higher concentrations of 4-OHE₁-1-N3Ade, 4-OHEI, and 4-OHE₁-1-N7Gua in comparison with that of 4-OHE₁-2-NAcCys provided similar recovery of the latter compound. Spectrum (c) shows another CE electropherogram obtained after a ten-fold buffer-diluted human urine sample was spiked with 4-OHE₁-2-NAcCys (c=10₋₆ M) and subsequently run through the affinity column. A remarkably simple CE electropherogram was obtained with the major peak (#2) corresponding to 4-OHE₁-2-NAcCys. The identification of this peak was confirmed by the standard spiking procedure. Again, an excellent recovery of ˜80% was obtained. Finally, curve d in FIG. 10 was obtained for the 4-OHE₁-2-NAcCys standard (c=10⁻⁴ M), and is shown for comparison. These data clearly demonstrate that very efficient recovery of analytes of interest can be obtained, which, in combination with the various separation and identification methods described herein, provide the means for analyzing human samples.

### Example 11

This example describes the detection of 4-OHE₁-2-NAcCys conjugates and 4-OHE -1-N3Ade adducts in urine of breast cancer patients.

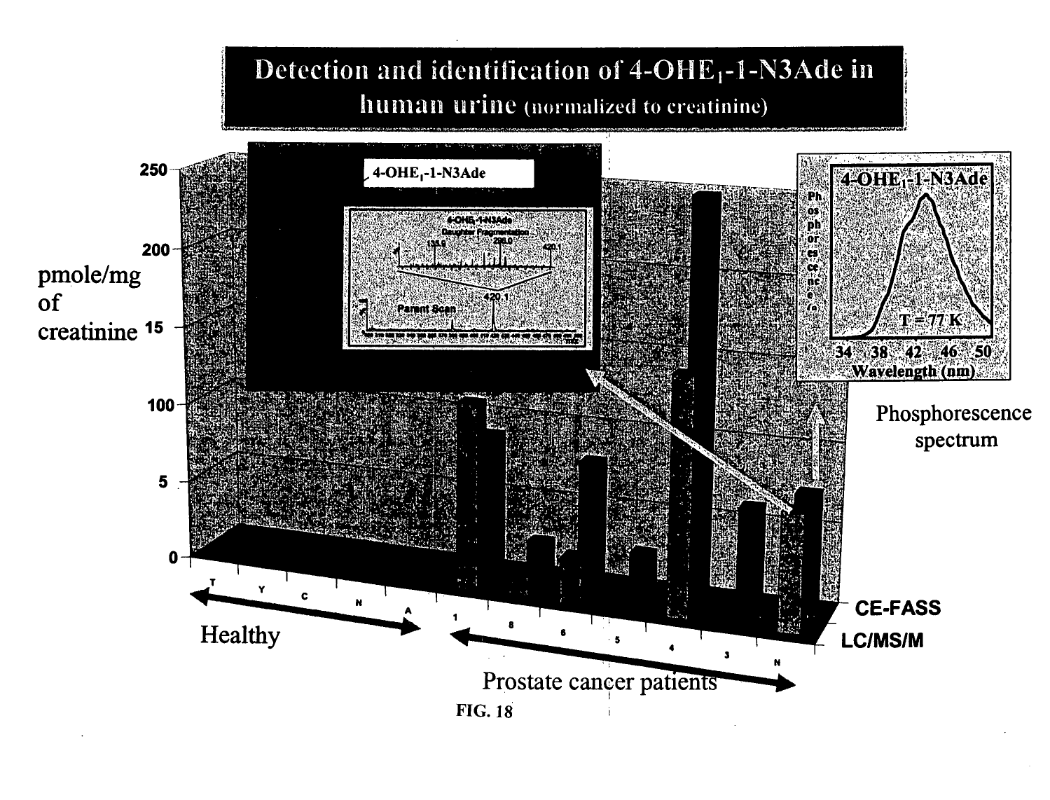

4-OHE₁-2-NAcCys and 4-OHE₁-1-N3Ade are present in urine obtained from a woman with breast carcinoma. An example of an absorbance based electropherogram obtained at 214 nm for a 20 mL urine sample run through the 2E₉ MAb based column and subsequently eluted for CE/FASS analysis is shown in curve (b) of FIG. 18A. As expected, only one major peak (#1) is observed, which corresponds to 4-OHE₁-2-NAcCys as proven by spectrum (a) obtained for the 4-OHE₁-2-NAcCys standard. Identification of this analyte was also confirmed by the standard spiking procedure and room T absorption spectra of peak #1. A similar procedure was used to identify the presence of 4-OHE-1-N3Ade in urine of the same patient; namely, spectra c, d, and e of frame B correspond to a urine sample from a breast cancer patient, 4-OHE₁-2-NAcCys conjugate standard, and urine from a healthy individual, respectively.

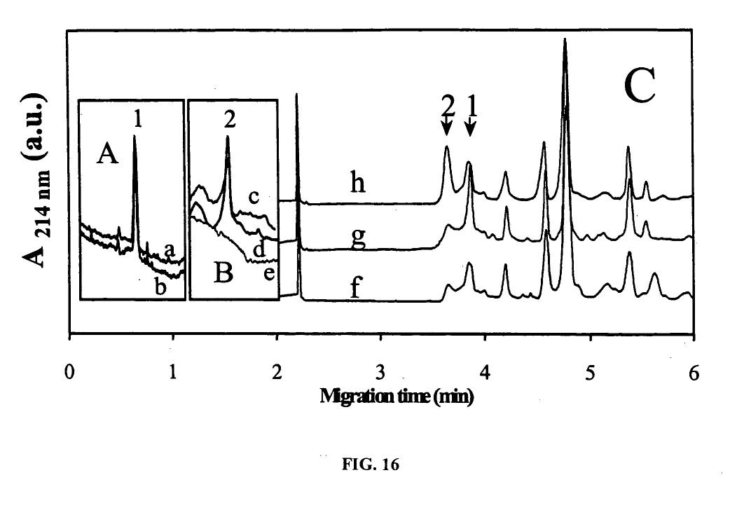

The migration time of the main peak in curve c is identical to that of peak #2 obtained with 4-OHE₁-2-NAcCys standard (curve d), clearly suggesting that this conjugate is excreted into urine of the breast cancer patient. Note that this conjugate is not observed in the urine sample from the healthy individual (see curve e). However, quantitation of the above analytes was impossible, as both MAb-columns were saturated, suggesting relatively high concentrations. This also is supported by the data shown in FIG. 16C; here, spectrum f is the electropherogram obtained with CE/FASS for the same methanol-extracted and pre-concentrated (by evaporation) urine sample. Curves g and h were obtained for the same urine extract after spiking with standards of 4-OHE₁-2-NAcCys (curve g) and 4-OHE₁-1-N3Ade (curve h), respectively. Comparison of curves f, g, and h indicates that peaks 1 and 2 correspond to the 4-OHE₁-2-NAcCys and 4-OHE₁-1-N3Ade adduct, respectively. Preliminary quantitative data revealed that the concentration of 4-OHE₁-2-NAcCys and 4-OHE₁-1-N3Ade in this urine sample is about 10-6 and 2.5×10⁻⁷ M, respectively. We hasten to add that so far none of the above two analytes has been identified in urine samples (three samples were studied) from healthy men and women, suggesting that both 4-OHE₁-2-NAcCys and 4-OHE₁-1-N3Ade could constitute excellent biomarkers of breast cancer risk.

### Example 12

This example describes the detection of CEQ-derived DNA adducts in urine of a prostate cancer patient.

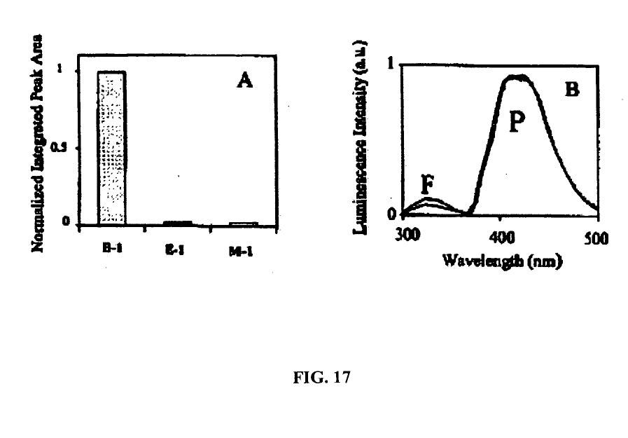

Urine samples (20 mL each; provided by Dr. David Matthews, a physician in Charlotte, N.C.) from three men were analyzed in blind studies using different detection methods. The question was asked whether any of these individuals forms 4-OHE₁(E₂)-DNA adducts. The first sample (B-1) was from a 55-yr-old male with prostate cancer (two yrs past prostatectomy), with initially very low PSA levels post-operation, which are now elevated; this patient is currently undergoing radiation therapy. The urine samples labeled E-1 and M-1 were obtained from a 40 yr old male with Paget's disease of the scrotum and a healthy male, respectively. Urine sample were analyzed using affinity column purification, i.e., the adducts of interest were pulled out of urine samples using home built columns equipped with the 15 GB MAb specifically developed for the 4-OHE₁(E₂)-1-N3Ade adducts. Eluted pre-concentrated samples were separated by CE with FASS, and absorbance electropherograms were collected. Adduct identification was accomplished by a standard spiking procedure with synthesized DNA adduct standard. The bars in frame A of FIG. 17 correspond to the integrated (normalized) area of the electropherogram peaks assigned to 4-OHE₁(E₂)-1-N3Ade. Only the sample from the prostate cancer patient contained a large amount of 4-OHE₁(E₂)-1-N3Ade (c=2.2×10⁻⁷ M normalized to creatinine concentration). The identity of this adduct was confirmed by low-temperature (77K) luminescence spectroscopy, as shown in frame B of FIG. 17; the solid lines with and without blue dots correspond to the adduct standard and the analyte eluted from the 15 GB-MAb based column. The spectra are nearly indistinguishable, proving that the eluted analyte corresponds to 4-OHE₁(E₂)-1-N3Ade. The amount of this adduct in samples E-1 and M-1 was slightly above background (i.e. ˜4×10⁻⁹ M). These findings were also confirmed by another approach; namely, urine samples after lyophilization and methanol extraction were pre-concentrated and analytes therein were separated by CE/FASS with absorbance detection. Thus, 4-OHE₁(E₂)-1-N3Ade is being excreted into urine of the prostate cancer patient. Therefore, CEQ-derived DNA adducts (and/or CEO-derived conjugates) can serve as biomarkers to investigate the hypothesis that metabolically activated endogenous estrogens are involved in the initiation of prostate and breast (Markushin et al., Chem. Res. Toxicol. 16:1107 (2003)) cancers.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A method for detecting a biological marker in a sample, which comprises a complex mixture of molecules, from a patient, which method comprises:
exposing a detection site having bound monoclonal antibodies specific for the biological marker to the sample;
washing the detection site with a solution that removes substantially unbound molecules from the detection site;
exposing the detection site to a reporter molecule, which is substantially identical to the biological marker, wherein the reporter molecule is detectably-labeled;
washing the detection site with a solution that removes substantially unbound reporter molecules from the detection site; and
assessing the degree of binding at the detection site by the reporter molecule, wherein a high degree of binding by the reporter molecule is indicative of an absence or a low concentration of the biological marker in the sample, and wherein an absence or a low degree of binding by the reporter molecule is indicative of a high concentration or moderate concentration of the biological marker in the sample.

2. The method of claim 1, wherein the biological marker is a conjugate (and/or DNA adduct) derived from catechol estrogen quinone.

3. A reporter molecule selected from the group consisting of a 4-OHE₁-2-N-AcCys conjugate, a 4-OHE₂-2-N-AcCys conjugate, a 4-OHE₁-1-N3 Ade adduct, and a 4-OHE₂-1-N3 Ade adduct, and wherein the reporter molecule is detectably-labeled.

4. A monoclonal antibody having specificity for 4-OHE₁-2-N-AcCys and 4-OHE₂-2-N-AcCys conjugates.

5. A monoclonal antibody having specificity for 4-OHE₁-1-N3 Ade and 4-OHE₂-1-N3 Ade and/or 4-OHE₁-1-N7 Gua and 4-OHE₂-1-N7 Gua adducts.

6. A hapten selected from the group consisting of 4-OHE₁/E₂-2-NAcCys-16-MCC, 4-OH-17AM-E₂-2-NAcCys-MCC, and 4-OH-17-AM-E₂-1-N3Ade-MCC, any one of which is optionally labeled with a detectable label.

7. A biochip comprising a monoclonal antibody having specificity for a conjugate and/or a DNA adduct derived from CEQ.

8. A kit comprising the biochip of claim 7, a hapten, and a reporter molecule.
